(19) United States
(12) Patent Application Publication
(10) Pub. No.: US 2024/0002956 A1
(43) Pub. Date: Jan. 4, 2024

(54) SENSITIVE QUANTITATIVE DETECTION OF SARS-CoV-2 USING DIGITAL WARM-START CRISPR ASSAY

(71) Applicant: UNIVERSITY OF CONNECTICUT, Farmington, CT (US)

(72) Inventors: XIONG DING, Farmington, CT (US); CHANGCHUN LIU, Farmington, CT (US)

(21) Appl. No.: 18/037,361

(22) PCT Filed: Nov. 17, 2021
(86) PCT No.: PCT/US2021/059727

§ 371 Date: May 17, 2023

Related U.S. Application Data
(60) Provisional application No. 63/115,291, filed on Nov. 18, 2020.

(51) Int. Cl. C12Q 1/6844; C12N 9/22

(52) U.S. Cl. CPC C12Q 1/6844; C12N 9/22; C12N 2310/20

(57) ABSTRACT

The disclosure provides materials and methods for detection of pathogens. In particular, the disclosure provides primers and compositions for detection of viral pathogens, such as SARS-CoV-2. In addition, the disclosure provides a warm-start digital CRISPR assay for detection of viral pathogens, including SARS-CoV-2.

### STATEMENT REGARDING RELATED APPLICATIONS

This application claims priority to U.S. Provisional Patent Application No. 63/115,291, filed Nov. 18, 2020, the entire contents of which are incorporated herein by reference for all purposes.

### STATEMENT REGARDING FEDERAL FUNDING

This invention was made with government support under Grant No. R01EB023607, R61AI154642, and R01CA214072 awarded by the National Institutes of Health. The government has certain rights in the invention.

### FIELD

The present invention relates to methods for detection of pathogens. In particular, the disclosure relates to methods for detection of SARS-CoV-2 using a warm-start CRISPR assay.

### BACKGROUND

Since its emergence in December 2019 (1), severe acute respiratory syndrome coronavirus 2 (SARS-CoV-2) has spread worldwide, resulting in over one million deaths (2). As of now, fully validated vaccines and antiviral drugs are still unavailable. Therefore, methods for sensitive detection of this deadly virus and for quantification of viral loads in infected subjects are of upmost importance.

To detect and quantify SARS-CoV-2, TaqMan probe-based reverse transcription polymerase chain reaction (RT-PCR) is frequently used due to strong sensitivity and specificity (3, 4). However, this gold standard method greatly depends on expensive real-time quantitation PCR instruments and its quantitation accuracy is highly associated with well-designed TaqMan probes, not suitable for small clinics or community health settings. Alternatively, some isothermal nucleic acid amplification methods have been developed to rapidly detect SARS-CoV-2, such as reverse transcription loop-mediated isothermal amplification (RT-LAMP) (5-7), reverse transcription recombinase polymerase amplification (RT-RPA) (8, 9), reverse transcription recombinase-aided amplification (RT-RAA) (10, 11), and sensitive splint-based one-pot isothermal RNA detection (SENSR) (12). However, these isothermal methods are inclined to qualitative detection or challenged by uncontrollable nonspecific amplification signals. Accordingly, what is needed are improved methods for accurate and sensitive detection of SARS-CoV-2 that are suitable for use in a variety of settings.

### SUMMARY

TO BE COMPLETED BY CASIMIR JONES UPON FINALIZATION OF THE CLAIMS

Other aspects and embodiments of the disclosure will be apparent in light of the following detailed description and accompanying figures.

### BRIEF DESCRIPTION OF THE DRAWINGS

FIG. 1A-1B shows an overview of digital warm-start CRISPR (WS-CRISPR) assay. (FIG. 1A) One-pot WS-CRISPR reaction mixture is first prepared in one tube. After distribution into QuantStudio 3D digital chip, over ten thousand sub-nanoliter (˜0.7 nL) microreactions are isolated in microwells. When incubated at 52° C., each microreaction with SARS-CoV-2 RNA target undertakes WS-CRISPR reaction and generates strong green fluorescence (positive spots), whereas not in those without target (negative spots). Scale bar is 300 m. Through detecting and counting the positive microreactions (or spots), SARS-CoV-2 RNA can be quantified based on the proportion of positive spots. (FIG. 1B) Working principle of one-pot WS-CRISPR assay for SARS-CoV-2 detection. The working principle in a single microwell (shown in the bottom right of FIG. 1A) is shown. The WS-CRISPR reaction mixture contains Cas12a-crRNA complex, six DAMP primers (two outer primers of FO and RO, two inner primers of FI and RI, and two competition primers of FI and RI), ssDNA-FQ reporter, SuperScript IV reverse transcriptase, Bst DNA polymerase, pyrophosphatase (PPase) and SARS-CoV-2 RNA in a “one-pot” format.

FIG. 2A-2D. Overcoming challenges for the one-pot WS-CRISPR assay. (FIG. 2A) Comparison of two different Cas12a nucleases in one-pot WS-CRISPR assay at different magnesium ion Mg²⁺ concentration. Lba Cas12a, EnGen Lba Cas12a from Lachnospiraceae bacterium ND2006 (New England Biolabs). A.s. Cas12a, Alt-R Cas12a (Cpf1) Ultra nuclease from Recombinant Acidaminococcus sp. BV3L6 (Integrated DNA Technologies). The used targets were 1 μM of synthetic SARS-CoV-2 N DNA fragment. Three replicates were run (n=3). (FIG. 2B) The chemical reaction process of the generation and degradation of the magnesium pyrophosphate precipitate due to the existence of pyrophosphatase during DNA polymerization. (FIG. 2C) Real-time fluorescence detection and endpoint fluorescence comparison of one-pot WS-CRISPR assay with PPase and/or PS primers at 52° C. PS primers specifically denote two phosphorothioated inner primers of FI and RI and the rest of primers are non-phosphorothioated. “w/o PS primers” means reactions with the non-phosphorothioated inner primers. Positive, the reaction with 5×10⁴ copies/μl SARS-CoV-2 RNA. Three replicates were run (n=3). (FIG. 2D) Effect of reaction temperature on the one-pot WS-CRISPR assay. Positive 1 and 2, the reactions with 3×10⁶ and 5×10⁴ copies/μl SARS-CoV-2 RNA, respectively. Three independent assays were conducted with the similar results. NTC, non-template control. Error bars represent the means±standard deviation (s.d.) from replicates. The statistical significance was analyzed using unpaired two-tailed t-test.

FIG. 3A-3C. Specificity and sensitivity of one-pot WS-CRISPR assay for SARS-CoV-2 detection. (FIG. 3A) Evaluation of eight reactions with various components through endpoint imaging after 90-min incubation and real-time fluorescence detection. Target RNA, 5×10⁵ copies/μl SARS-CoV-2 RNA. RT-DAMP mix contains six primers, reverse transcriptase, and Bst DNA polymerase in one reaction buffer. (FIG. 3B) Specificity of real-time/endpoint one-pot WS-CRISPR assay for SARS-CoV-2 detection. Three replicates were run (n=3). (FIG. 3C) Sensitivity of real-time/endpoint WS-CRISPR assay when detecting various concentrations of SARS-CoV-2 RNA. Six replicates were run (n=6). NTC, non-template control. Horizontal dashed lines indicate the cutoff fluorescence defined as the average intensity of NTC plus three times of the standard deviation. Error bars represent the means±s.d. from replicates. The statistical significance was analyzed using unpaired two-tailed t-test.

FIG. 4A-4E. Digital WS-CRISPR assay for SARS-CoV-2 detection. (FIG. 4A) A typical workflow of digital WS-CRISPR assay to detect SARS-CoV-2 in clinical samples. (FIG. 4B) Endpoint fluorescence micrographs of the QuantStudio digital chip for the SARS-CoV-2 detection with various incubation time (0, 10, 30, 60, 90 and 120 min) at 52° C. In this digital WS-CRISPR assay, 1×10⁶ copies/μl SARS-CoV-2 RNA was loaded. (FIG. 4C) The percentage of positive spots comparison for the digital WS-CRISPR assays with various incubation time at 52° C. The number of positive spots was counted by setting the same threshold in the ImageJ software. Percentages of positive spots in each micrograph was calculated (n=6). Error bars represent the means±s.d. from replicates. The statistical significance was analyzed using unpaired two-tailed t-test. (FIG. 4D) Workflow to test effect of various waiting times on digital on results. (FIG. 4E) Effect of various waiting time at room temperature on digital WS-CRISPR assay and digital RT-AIOD-CRISPR assay during one-pot reaction preparation and distribution steps. After specific waiting time, the chips were directly observed without incubation. Positive, the reaction with 5×10⁵ copies/μl SARS-CoV-2 RNA. NTC, non-template control. Scale bars are 300 m. Each micrograph is a representative of six distinct regions taken to cover about 2809 microreactions.

FIG. 5A-5C. Specificity and sensitivity of the digital WS-CRISPR assay for SARS-CoV-2 detection. a, Endpoint fluorescence micrographs of the QuantStudio digital chip for the digital WS-CRISPR assays with non-target nucleic acids. SARS-CoV-2 PC, SARS-CoV control, MERS-CoV control, and Hs_RPP30 PC were from the Integrated DNA Technologies. b, Endpoint fluorescence micrographs of the chip for the digital WS-CRISPR assays testing various concentrations of SRS-CoV-2 RNA within 90-min incubation at 52° C. c, The linear relationship between percentage of positive spots and concentration of targets. The number of positive spots was counted by setting the same threshold in the ImageJ software. For each concentration's testing, total positive spots in all the six micrographs were used and three chips were taken to run three independent assays (n=3). Each micrograph is a representative of six distinct regions taken to cover about 2809 microreactions. The number of positive spots were counted by setting the same threshold in the ImageJ software. Scale bars are 300 m. Error bars represent the means±s.d. from replicates.

FIG. 6A-6B. Clinical validation of digital WS-CRISPR assay. (FIG. 6A) Endpoint fluorescence micrographs of the QuantStudio digital chip for detecting SARS-CoV-2 RNA extracted from 32 clinical swab samples (S1-S32) and 3 saliva samples (Saliva S1-S3). The indicated Cq values were the results of RT-qPCR assays. (FIG. 6B) Heat map displaying the determined SARS-CoV-2 RNA concentration by RT-qPCR and digital WS-CRISPR for each sample. The presented concentrations are the average values in three independent assays. Each micrograph is a representative of six distinct regions taken to cover about 2809 microreactions. PC, SARS-CoV-2-positive control sample. NC, SARS-CoV-2-negative control sample. NTC, non-template control. Scale bars are 300 m.

FIG. 7A-7B. Direct detection of SARS-CoV-2 in crude saliva samples by digital WS-CRISPR assay. (FIG. 7A) Workflow for the direct SARS-CoV-2 testing in spiked saliva samples by digital WS-CRISPR assay. (FIG. 7B) Endpoint fluorescence micrographs of the chip for direct detection of the SARS-CoV-2 virus spiked in saliva samples. Saliva samples 1-5, the samples with 10%, 5%, 2.5%, 1%, and 0% of heat-inactivated SARS-CoV-2 virus. Each micrograph is a representative of six distinct regions taken to cover about 2809 microreactions. PC, SARS-CoV-2-positive control sample. NC, SARS-CoV-2-negative control sample. NTC, non-template control. Scale bars are 300 m.

FIG. 8. Effect of pyrophosphatase (PPase) concentrations on one-pot WS-CRISPR assay. Concentrations of PPase are indicated above the respective graphs. Results are summarized in the bottom right bar graph. Positive, the reaction with 5×10⁴ copies/μl SARS-CoV-2 RNA. NTC, non-template control. Three replicates were run (n=3). Error bars represent the means±standard deviation (s.d.) from replicates.

FIG. 9. Effect of magnesium ion (Mg²⁺) concentration on one-pot WS-CRISPR assay. Concentrations of magnesium ion are indicated above the respective graphs. Results are summarized in the bottom right bar graph. Positive, the reaction with 5×10⁵ copies/μl SARS-CoV-2 RNA. NTC, non-template control. Three replicates were run (n=3). Error bars represent the means±standard deviation (s.d.) from replicates.

FIG. 10. Effect of Bst DNA polymerase (large fragment) concentration on one-pot WS-CRISPR assay. Concentrations of polymerase are indicated above the respective graphs. Results are summarized in the bar graph. Positive, the reaction with 5×10⁴ copies/μl SARS-CoV-2 RNA. NTC, non-template control. Three replicates were run (n=3). Error bars represent the means±standard deviation (s.d.) from replicates.

FIG. 11. Effect of SuperScript IV reverse transcriptase concentration on one-pot WS-CRISPR assay. Concentrations of transcriptase are indicated above the respective graphs. Results are summarized in the bar graph. Positive, the reaction with 5×10⁴ copies/μl SARS-CoV-2 RNA. NTC, non-template control. Three replicates were run (n=3). Error bars represent the means±standard deviation (s.d.) from replicates.

FIG. 12A-12B. Effect of different DNA polymerases on one-pot WS-CRISPR assay. Positive, the reaction with 5×10⁴ copies/μl SARS-CoV-2 RNA. NTC, non-template control. FIG. 12A shows graphs of results from experiments using the following polymerases: Bst LF (Bst DNA polymerase (large fragment)), Bst 2.0 (Bst 2.0 DNA polymerase), Bst 3.0 (Bst 3.0 DNA polymerase), and GspSSD 2.0 (GspSSD 2.0 DNA polymerase). FIG. 12B shows graphs of results from experiments using the following polymerases: Bsm LF (Bsm DNA polymerase (large fragment)), IsoPol BST⁺ (IsoPol BST⁺ DNA polymerase), IsoPol SD⁺ (IsoPol SD⁺ DNA polymerase). Results are summarized in the bottom right panel of FIG. 12B. Three replicates were run (n=3). Error bars represent the means±standard deviation (s.d.) from replicates.

FIG. 13A-13B. Real-time RT-qPCR assay of the tenfold serial dilution of SARS-CoV-2 RNA. (FIG. 13A) Real-time fluorescence curves of the RT-qPCR assay. (FIG. 13B) A four-point calibration curve developed to quantify the amount of SARS-CoV-2 RNA extracted from clinical samples. NTC, non-template control. Three replicates were run (n=3). Error bars represent the means±standard deviation (s.d.) from replicates.

### DETAILED DESCRIPTION OF THE INVENTION

### 1. Definitions

To facilitate an understanding of the present technology, a number of terms and phrases are defined below. Additional definitions are set forth throughout the detailed description.

As used herein, the term “about,” when referring to a value or to an amount is meant to encompass variations of within +20% from that value or amount. In some embodiments, “about” refers to ±20%, +10%, ±5%, +1%, ±0.5%, or 0.1% from the specified amount, as such variations are appropriate.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific and technical terms used in connection with the present disclosure shall have the meanings that are commonly understood by those of ordinary skill in the art. For example, any nomenclature used in connection with, and techniques of, cell and tissue culture, biochemistry, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “amino acid” refers to natural amino acids, unnatural amino acids, and amino acid analogs, all in their D and L stereoisomers, unless otherwise indicated, if their structures allow such stereoisomeric forms.

Natural amino acids include alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), Lysine (Lys or K), methionine (Met or M), phenylalanine (Phe or F), proline (Pro or P), serine (Ser or S), threonine (Thr or T), tryptophan (Trp or W), tyrosine (Tyr or Y) and valine (Val or V).

Unnatural amino acids include, but are not limited to, azetidinecarboxylic acid, 2-aminoadipic acid, 3-aminoadipic acid, beta-alanine, naphthylalanine (“naph”), aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, tertiary-butylglycine (“tBuG”), 2,4-diaminoisobutyric acid, desmosine, 2,2′-diaminopimelic acid, 2,3-diaminopropionic acid, N-ethylglycine, N-ethylasparagine, homoproline (“hPro” or “homoP”), hydroxylysine, allo-hydroxylysine, 3-hydroxyproline (“3Hyp”), 4-hydroxyproline (“4Hyp”), isodesmosine, allo-isoleucine, N-methylalanine (“MeAla” or “Nime”), N-alkylglycine (“NAG”) including N-methylglycine, N-methylisoleucine, N-alkylpentylglycine (“NAPG”) including N-methylpentylglycine. N-methylvaline, naphthylalanine, norvaline (“Norval”), norleucine (“Norleu”), octylglycine (“OctG”), ornithine (“Orn”), pentylglycine (“pG” or “PGly”), pipecolic acid, thioproline (“ThioP” or “tPro”), homoLysine (“hLys”), and homoArginine (“hArg”).

As used herein, the term “artificial” refers to compositions and systems that are designed or prepared by man, and are not naturally occurring. For example, an artificial peptide or nucleic acid is one comprising a non-natural sequence (e.g., a nucleic acid or a peptide without 100% identity with a naturally-occurring protein or a fragment thereof).

As used herein, a “conservative” amino acid substitution refers to the substitution of an amino acid in a peptide or polypeptide with another amino acid having similar chemical properties, such as size or charge. For purposes of the present disclosure, each of the following eight groups contains amino acids that are conservative substitutions for one another:
1) Alanine (A) and Glycine (G);
2) Aspartic acid (D) and Glutamic acid (E);
3) Asparagine (N) and Glutamine (Q);
4) Arginine (R) and Lysine (K);
5) Isoleucine (I), Leucine (L), Methionine (M), and Valine (V);
6) Phenylalanine (F), Tyrosine (Y), and Tryptophan (W);
7) Serine (S) and Threonine (T); and
8) Cysteine (C) and Methionine (M).

Naturally occurring residues may be divided into classes based on common side chain properties, for example: polar positive (or basic) (histidine (H), lysine (K), and arginine (R)); polar negative (or acidic) (aspartic acid (D), glutamic acid (E)); polar neutral (serine (S), threonine (T), asparagine (N), glutamine (Q)); non-polar aliphatic (alanine (A), valine (V), leucine (L), isoleucine (I), methionine (M)); non-polar aromatic (phenylalanine (F), tyrosine (Y), tryptophan (W)); proline and glycine; and cysteine. As used herein, a “semi-conservative” amino acid substitution refers to the substitution of an amino acid in a peptide or polypeptide with another amino acid within the same class.

In some embodiments, unless otherwise specified, a conservative or semi-conservative amino acid substitution may also encompass non-naturally occurring amino acid residues that have similar chemical properties to the natural residue. These non-natural residues are typically incorporated by chemical peptide synthesis rather than by synthesis in biological systems. These include, but are not limited to, peptidomimetics and other reversed or inverted forms of amino acid moieties. Embodiments herein may, in some embodiments, be limited to natural amino acids, non-natural amino acids, and/or amino acid analogs.

Non-conservative substitutions may involve the exchange of a member of one class for a member from another class.

The term “amino acid analog” refers to a natural or unnatural amino acid where one or more of the C-terminal carboxy group, the N-terminal amino group and side-chain functional group has been chemically blocked, reversibly or irreversibly, or otherwise modified to another functional group. For example, aspartic acid-(beta-methyl ester) is an amino acid analog of aspartic acid; N-ethylglycine is an amino acid analog of glycine; or alanine carboxamide is an amino acid analog of alanine. Other amino acid analogs include methionine sulfoxide, methionine sulfone, S-(carboxymethyl)-cysteine, S-(carboxymethyl)-cysteine sulfoxide and S-(carboxymethyl)-cysteine sulfone.

The terms “complementary” and “complementarity” refer to the ability of a nucleic acid to form hydrogen bond(s) with another nucleic acid sequence by either traditional Watson-Crick base-paring or other non-traditional types of pairing. The degree of complementarity between two nucleic acid sequences can be indicated by the percentage of nucleotides in a nucleic acid sequence which can form hydrogen bonds (e.g., Watson-Crick base pairing) with a second nucleic acid sequence (e.g., 50%, 60%, 70%, 80%, 90%, and 100% complementary). Two nucleic acid sequences are “perfectly complementary” if all the contiguous nucleotides of a nucleic acid sequence will hydrogen bond with the same number of contiguous nucleotides in a second nucleic acid sequence. Two nucleic acid sequences are “substantially complementary” if the degree of complementarity between the two nucleic acid sequences is at least 60% (e.g., 65%, 70%, 75%, 80%, 85%, 90%, 95%. 97%, 98%, 99%, or 100%) over a region of at least 8 nucleotides (e.g., 9, 10, 11, 12, 13, 14, 15, 16, 17, 18, 19, 20, 21, 22, 23, 24, 25, 30, 35, 40, 45, 50, or more nucleotides), or if the two nucleic acid sequences hybridize under at least moderate, preferably high, stringency conditions. Exemplary moderate stringency conditions include overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C., or substantially similar conditions, e.g., the moderately stringent conditions described in Sambrook et al., infra. High stringency conditions are conditions that use, for example (1) low ionic strength and high temperature for washing, such as 0.015 M sodium chloride/0.0015 M sodium citrate/0.1% sodium dodecyl sulfate (SDS) at 50° C., (2) employ a denaturing agent during hybridization, such as formamide, for example, 50% (v/v) formamide with 0.1% bovine serum albumin (BSA)/0.1% Ficoll/0.1% polyvinylpyrrolidone (PVP)/50 mM sodium phosphate buffer at pH 6.5 with 750 mM sodium chloride and 75 mM sodium citrate at 42° C., or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at (i) 42° C. in 0.2×SSC, (ii) 55° C. in 50% formamide, and (iii) 55° C. in 0.1×SSC (preferably in combination with EDTA). Additional details and an explanation of stringency of hybridization reactions are provided in, e.g., Sambrook et al., Molecular Cloning: A Laboratory Manual, 3rd ed., Cold Spring Harbor Press, Cold Spring Harbor, N.Y. (2001); and Ausubel et al., Current Protocols in Molecular Biology, Greene Publishing Associates and John Wiley & Sons, New York (1994).

The terms “crRNA” or “CRISPR RNA” are used interchangeably herein. The term crRNA is used in the broadest sense to cover any RNA involved in CRISPR methods, including pre-crRNA, tracrRNA, and guide RNA.

The “guide RNA,” “single guide RNA,” and “synthetic guide RNA,” are used interchangeably herein and refer to a nucleic acid comprising a crRNA containing a guide sequence. The terms “guide sequence,” “guide,” and “spacer,” are used interchangeably herein and refer to the about 20 nucleotide sequence within a guide RNA that specifies the target site. In CRISPR/Cas systems, the guide RNA contains an approximate 20-nucleotide guide sequence followed by a protospacer adjacent motif (PAM) that directs the endonuclease via Watson-Crick base pairing to a target sequence.

As used herein, a “nucleic acid” or a “nucleic acid sequence” refers to a polymer or oligomer of pyrimidine and/or purine bases, preferably cytosine, thymine, and uracil, and adenine and guanine, respectively. The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41(14): 4503-4510 (2002)) and U.S. Pat. No. 5,034,506, incorporated herein by reference), locked nucleic acid (LNA; see Wahlestedt et al., Proc. Natl. Acad. Sci. U.S.A., 97: 5633-5638 (2000), incorporated herein by reference), cyclohexenyl nucleic acids (see Wang, J. Am. Chem. Soc., 122: 8595-8602 (2000), incorporated herein by reference), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “peptide” or “polypeptide” is a linked sequence of two or more amino acids linked by peptide bonds. The peptide or polypeptide can be natural, synthetic, or a modification or combination of natural and synthetic. Polypeptides include proteins such as binding proteins, receptors, and antibodies. The proteins may be modified by the addition of sugars, lipids or other moieties not included in the amino acid chain. The terms “polypeptide” and “protein,” are used interchangeably herein.

As used herein, the term “percent sequence identity” refers to the percentage of nucleotides or nucleotide analogs in a nucleic acid sequence, or amino acids in an amino acid sequence, that is identical with the corresponding nucleotides or amino acids in a reference sequence after aligning the two sequences and introducing gaps, if necessary, to achieve the maximum percent identity. Hence, in case a nucleic acid according to the technology is longer than a reference sequence, additional nucleotides in the nucleic acid, that do not align with the reference sequence, are not taken into account for determining sequence identity. Methods and computer programs for alignment are well known in the art, including BLAST, Align 2, and FASTA.

The term “sample” is used herein in the broadest sense and refers to any suitable sample, including liquids, solids, and gases. In some embodiments, the sample is a biological sample (e.g. a sample obtained from a subject). The biological sample may comprise a fluid sample or a tissue sample. In some embodiments, the biological sample is a blood sample or a blood product such as serum or plasma. In some embodiments, the sample comprises urine. In embodiments, the sample is a respiratory specimen, including a nasal sample (e.g. a nasal swab), a nasopharyngeal sample (e.g. a nasopharyngeal swab), an oropharyngeal sample (e.g. an oropharyngeal swab), a mid-turbinate sample (e.g. a mid-turbinate swab), sputum, endotracheal aspirate or bronchoalveolar lavage. In some embodiments, the sample is a cerebrospinal fluid sample. In some embodiments, the sample is a saliva sample. In some embodiments, the sample is a tissue sample. In some embodiments, the sample is obtained from a subject suspected of having a viral infection. In some embodiments, the sample is obtained from a subject suspected of having an upper respiratory infection. In some embodiments, the sample is obtained from a subject suspected of having a SARS-CoV-2 infection. In some embodiments, the subject is a human. The sample can be used directly as obtained from a patient or can be pre-treated, such as by heating, filtration, distillation, extraction, concentration, centrifugation, inactivation of interfering components, addition of reagents, and the like.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In some embodiments, the subject is a human.

The terms “target sequence,” “target nucleic acid,” and “target site” are used interchangeably herein to refer to a polynucleotide (nucleic acid, gene, chromosome, genome, etc.) to which a guide sequence (e.g., a guide RNA) is designed to have complementarity, wherein hybridization between the target sequence and a guide sequence promotes the formation of a Cas/CRISPR complex, provided sufficient conditions for binding exist. In some embodiments, the target sequence is a viral nucleic acid sequence. In some embodiments, the target sequence is a SARS-CoV-2 sequence.

### 2. Primers

In some aspects, provided herein are primers. In some embodiments, provided herein are primers for amplifying SARS-CoV-2 nucleic acid. In some embodiments, provided herein is a composition comprising a plurality of primers for amplifying SARS-CoV-2 nucleic acid.

In some embodiments, provided herein is a primer having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 1. In some embodiments, provided herein is a primer having an amino acid sequence set forth in SEQ ID NO: 1. In some embodiments, provided herein is a primer having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 2. In some embodiments, provided herein is a primer having an amino acid sequence set forth in SEQ ID NO: 2.

In some embodiments, provided herein is a primer having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 3. In some embodiments, provided herein is a primer having an amino acid sequence set forth in SEQ ID NO: 3. In some embodiments, provided herein is a primer having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 5. In some embodiments, provided herein is a primer having an amino acid sequence set forth in SEQ ID NO: 5.

In some embodiments, provided herein is a phosphorothioated primer. In some embodiments, provided herein is a primer having at least 90% sequence identity to SEQ ID NO: 4. In some embodiments, provided herein is a primer having an amino acid sequence set forth in SEQ ID NO: 4. In some embodiments, provided herein is a primer having at least 90% sequence identity to SEQ ID NO: 6. In some embodiments, provided herein is a primer having an amino acid sequence set forth in SEQ ID NO: 6.

In some embodiments, provided herein is a primer having at least 90% sequence identity to SEQ ID NO: 7. In some embodiments, provided herein is a primer having an amino acid sequence set forth in SEQ ID NO: 7. In some embodiments, provided herein is a primer having at least 90% sequence identity to SEQ ID NO: 8. In some embodiments, provided herein is a primer having an amino acid sequence set forth in SEQ ID NO: 8.

In some embodiments, provided herein is a composition comprising a primer having at least 90% sequence identity to SEQ ID NO: 1, a primer having at least 90% sequence identity to SEQ ID NO: 2, a primer having at least 90% sequence identity to SEQ ID NO: 4, a primer having at least 90% sequence identity to SEQ ID NO: 6, a primer having at least 90% sequence identity to SEQ ID NO: 7, and a primer having at least 90% sequence identity to SEQ ID NO: 8.

In some embodiments, provided herein is a composition comprising a primer having the amino acid sequence set forth in SEQ ID NO: 1, a primer having the amino acid sequence set forth in SEQ ID NO: 2, a primer having the amino acid sequence set forth in SEQ ID NO: 4, a primer having the amino acid sequence set forth in SEQ ID NO: 6, a primer having the amino acid sequence set forth in SEQ ID NO: 7, and a primer having the amino acid sequence set forth in SEQ ID NO: 8. The composition may further comprise additional reagents, including those described in the warm-start CRISPR reaction mixture of section 3. Suitable additional reagents include, for example, a Cas endonuclease, a target-specific crRNA, pyrophosphatase, a reverse transcriptase, a DNA polymerase, and the like, including the specific reagents and amounts thereof described below.

### 3. Assays

In some aspects, provided herein are assays. In some embodiments, provided herein is a digital warm-start assay for detecting a target in a sample. In some embodiments, the warm-start digital assay for detecting a target in a sample comprises contacting a sample with a warm-start CRISPR reaction mixture, partitioning the warm-start CRISPR reaction mixture into a plurality of microwells, amplifying the target, if present in the sample, and detecting a signal in each of the plurality of microwells. Detection of the signal in a given microwell indicates the presence of the target in the microwell.

In some embodiments, the warm-start digital assay for detecting a target in a sample comprises contacting providing a warm-start CRISPR reaction mixture and partitioning the warm-start CRISPR reaction mixture into a plurality of microwells, wherein each microwell comprises a sample. In some embodiments, the assay further comprises amplifying the target, if present in the sample, and detecting a signal in each of the plurality of microwells. Detection of the signal in a given microwell indicates the presence of the target in the microwell.

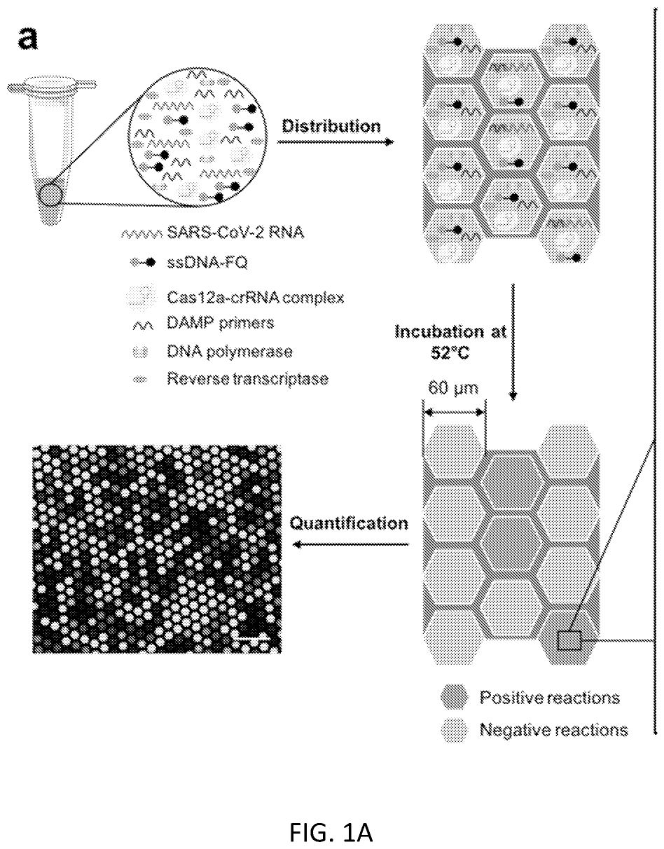

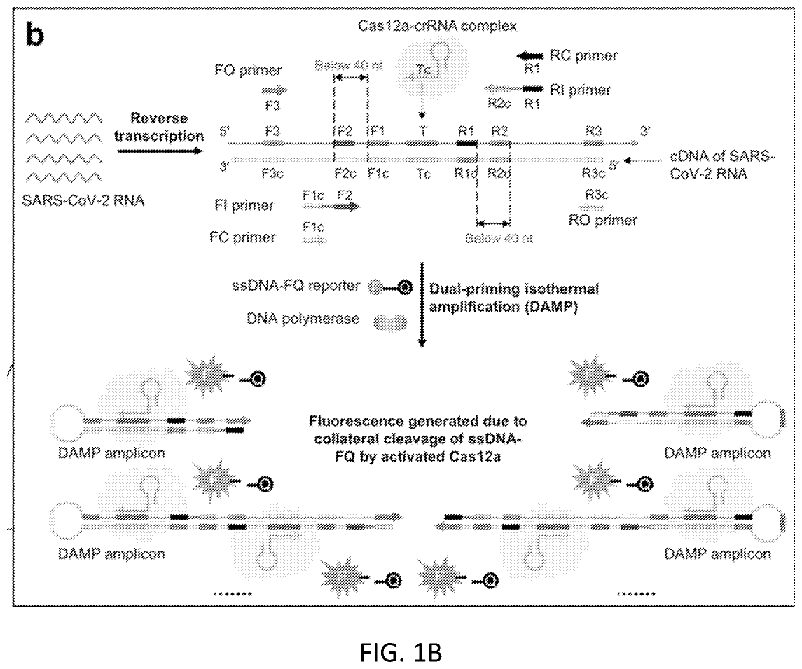

In some embodiments, the target is amplified by isothermal amplification. In some embodiments, the assays described herein take advantage of isothermal amplification techniques to amplify the target, if present in the sample, and CRISPR/Cas technology to detect the target, if present. Accordingly, in some embodiments the warm-start CRISPR reaction mixture comprises a Cas endonuclease and a target-specific crRNA. The Cas endonuclease may be Cas9, Cas12a (Cpf1), or Cas13. In some embodiments, the endonuclease is Cas12a. The target-specific crRNA depends on the intended target to be detected in the sample. Generally speaking, the Cas endonuclease (e.g. Cas12a) and the target-specific crRNA form a complex. For example, as shown in FIG. 1B the Cas12a and the target-specific crRNA form a complex, referred to as a Cas12a-crRNA complex. The complex binds to a target sequence on a nucleic acid, and induces selective cleavage, which generates a fluorescent signal that can be detected and/or measured to determine the presence of the target in the sample. In some embodiments, the crRNA comprises a guide RNA sequence (gRNA) that directs the endonuclease-crRNA complex (e.g. the Cas12a-crRNA complex) to bind to the desired sequence.

In some embodiments, the target is a nucleic acid. In some embodiments, the target is nucleic acid from a pathogen. For example, in some embodiments the target is viral nucleic acid. For example, the target may be viral RNA. In some embodiments, the target comprises viral nucleic acid from a virus causing upper respiratory infection. In some embodiments, the target comprises viral nucleic acid from an upper respiratory pathogen selected from SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, aspergillus, Streptococcus. pyogenes.

In some embodiments, the target is SARS-CoV-2 nucleic acid. In some embodiments, the target-specific crRNA comprises a nucleotide sequence having at least 90% sequence identity to SEQ ID NO: 9. For example, in some embodiments the target-specific crRNA comprises a nucleotide sequence having at least 90%, at least 91%, at least 92%, at least 93%, at least 94%, at least 95%, at least 96%, at least 97%, at least 98%, at least 99%, or 100% sequence identity to SEQ ID NO: 9. In some embodiments, the target-specific crRNA comprises the nucleotide sequence of SEQ ID NO: 9.

In some embodiments, the warm-start CRISPR reaction mixture further comprises primers for amplification of the target. In some embodiments, the warm-start CRISPR reaction mixture comprises primers for isothermal amplification of the target. In some embodiments, the warm-start CRISPR reaction mixture comprises primers for dual-priming isothermal amplification of the target (DAMP). DAMP is described in Ding et al., Anal. Chem. (2019) 91:20; 12852-12858, the entire contents of which are incorporated herein by reference for all purposes. In some embodiments, the warm-start CRISPR reaction mixture comprises outer primers and inner primers. In some embodiments, the warm-start CRISPR reaction mixture comprises a forward outer primer and a reverse outer primer. In some embodiments, the outer primers are 10-40 contiguous nucleotides in length. For example, the forward outer primer and/or the reverse outer primer may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 contiguous nucleotides in length. In some embodiments, the warm-start CRISPR reaction mixture further comprises a forward inner primer and reverser inner primer. In some embodiments, the inner primers are 20-60 contiguous nucleotides in length. For example, in some embodiments the forward inner primer and/or the reverse inner primer are 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 contiguous nucleotides in length.

The primers are designed to amplify the target (e.g. the target nucleic acid sequence). Accordingly, appropriate selection of primers depends on the target itself. In some embodiments, the target is SARS-CoV-2. In some embodiments, the target is or is present within the nucleoprotein (N) gene of SARS-CoV-2. Accordingly, the primers may be designed to amplify the desired portion of the N gene of SARS-CoV-2. Other suitable primers may be designed for amplification of other desired targets. For example, the target may be viral nucleic acid, and the primers designed for amplification of said viral nucleic acid. For example, primers may be designed and used for amplification of viral nucleic acid selected from SARS-CoV2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, Aspergillus, and Streptococcus Pyogenes nucleic acid.

In some embodiments, the warm-start CRISPR reaction mixture comprises a forward outer primer and a reverse outer primer, wherein the forward outer primer comprises an nucleotide sequence having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 1 and wherein the reverse outer primer comprises an nucleotide sequence having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 2. In some embodiments, the forward outer primer comprises an nucleotide sequence set forth in SEQ ID NO: 1 and the reverse outer primer comprises an amino acids sequence set forth in SEQ ID NO: 2.

In some embodiments, the warm-start CRISPR reaction mixture comprises inner primers. In some embodiments, the warm-start CRISPR reaction mixture comprises a forward inner primer comprising a nucleotide sequence having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 3 and an outer primer comprising a nucleotide sequence having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 5.

In some embodiments, the forward inner primer and/or the reverse inner primer is phosphorothioated. In some embodiments, the forward inner primer and the reverse inner primer is phosphorothioated. A “phosphorothioated primer” or a primer that “is phosphorothioated” refers to a primer in which at least one nucleotide comprises a phosphorothioate modification. For example, a phosphorothioated primer may comprise at least one, at least two, at least three, at least four, at least five, at least six, at least seven, at least eight, at least nine, or at least 10 nucleotides that are a phosphorothioated. In some embodiments, the nucleotides that are phosphorothioated are contiguous. For example, the primer may contain at least two, at least three, at least four, at least five, at least six, at least seven, at least eight, at least nine, or at least 10 contiguous nucleotides that are a phosphorothioated. In some embodiments, a phosphorothioated primer comprises 11, 12, 13, 14, 15, 16, 17, 18, 19, 20, 21, 22, 23, 24, 25, 26, 27, 27, 29, or 30 nucleotides that are phosphorothioated. In some embodiments, the inner primers comprise a forward inner primer comprising a nucleotide sequence having at least 90% sequence identity to SEQ ID NO: 4, and the reverse inner primer comprises an nucleotide sequence having at least 90% sequence identity to SEQ ID NO: 6. In some embodiments, the forward inner primer comprises a nucleotide sequence set forth in SEQ ID NO: 4, and the reverse inner primer comprises an nucleotide sequence set forth in SEQ ID NO: 6.

In some embodiments, the warm-start CRISPR reaction mixture further comprises a forward competition primer and a reverse competition primer. The competition primers may be added to mediate pair-priming strand extension. In some embodiments, the competition primers overlap with the inner primers, but are shorter than the inner primers. For example, in some embodiments, the forward competition primer comprises at least 10 contiguous nucleotides also present within the sequence of the forward inner primer, wherein the forward competition primer is shorter than the forward inner primer. For example, in some embodiments the forward competition primer comprises 10, 11, 12, 13, 14, 15, 16, 17, 18, 19, 20, 21, 22, 23, 24, 25, 26, 27, 28, 29, or 30 contiguous nucleotides also present within the sequence of the forward inner primer. In some embodiments, the forward competition primer comprises 15-25 contiguous nucleotides present within the nucleotide sequence of the forward inner primer. In some embodiments, the forward competition primer comprises a nucleotide sequence having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 7. In some embodiments, the forward competition primer comprises the nucleotide sequence set forth in SEQ ID NO: 7.

In some embodiments, the reverse competition primer comprises at least 10 contiguous nucleotides also present within the sequence of the reverse inner primer, wherein the reverse competition primer is shorter than the reverse inner primer. For example, in some embodiments the reverse competition primer comprises 10, 11, 12, 13, 14, 15, 16, 17, 18, 19, 20, 21, 22, 23, 24, 25, 26, 27, 28, 29, or 30 contiguous nucleotides also present within the sequence of the reverse inner primer. In some embodiments, the reverse competition primer comprises 15-25 contiguous nucleotides present within the nucleotide sequence of the reverse inner primer. In some embodiments, the reverse competition primer comprises a nucleotide sequence having at least 90% sequence identity (e.g. at least 90%, at least 91%, at least 92%, at least 93%, at least 94%, at least 95%, at least 96%, at least 97%, at least 98%, at least 99%, or 100% sequence identity) to SEQ ID NO: 8. In some embodiments, the reverse competition primer comprises the nucleotide sequence set forth in SEQ ID NO: 8.

In some embodiments, the warm-start CRISPR reaction mixture further comprises pyrophosphatase. In some embodiments, the warm-start CRISPR reaction mixture comprises less than 0.4 U/μl pyrophosphatase (concentration relative to the total volume of the warm-start CRISPR reaction mixture, not including the sample). For example, in some embodiments the warm-start CRISPR reaction mixture comprises about 0.01 U/μl to about 0.39 U/μl pyrophosphatase. In some embodiments, the warm-start CRISPR reaction mixture comprises less than about 0.39 U/μl, less than about 0.38 U/μl, less than about 0.37 U/μl, less than about 0.36 U/μl, less than about 0.35 U/μl, less than about 0.34 U/μl, less than about 0.33 U/μl, less than about 0.32 U/μl, less than about 0.31 U/μl, or less than about 0.3 U/μl pyrophosphatase. In some embodiments, the warm-start CRISPR reaction mixture comprises about 0.29 U/μl, about 0.28 U/μl, about 0.27 U/μl, about 0.26 U/μl, about 0.25 U/μl, about 0.24 U/μl, about 0.23 U/μl, about 0.22 U/μl, about 0.21 U/μl, about 0.2 U/μl pyrophosphatase. In some embodiments, the warm-start CRISPR reaction mixture comprises about 0.2 U/μl pyrophosphatase.

In some embodiments, the warm-start CRISPR reaction mixture further comprises a DNA polymerase. In some embodiments, the DNA polymerase comprises Bst DNA polymerase. In some embodiments, the warm-start CRISPR reaction mixture comprises less than about 36 U/μl (concentration relative to the total volume of the warm-start CRISPR reaction mixture, not including the sample) of the DNA polymerase (e.g. Bst DNA polymerase). In some embodiments, the warm-start CRISPR reaction mixture comprises about 1 U/μl to about 35 U/μl of the DNA polymerase (e.g. Bst DNA polymerase). In some embodiments, the warm-start CRISPR reaction mixture comprises about 1 U/μl, about 2 U/μl, about 3 U/μl, about 4 U/μl, about 5 U/μl, about 6 U/μl, about 7 U/μl, about 8 U/μl, about 9 U/μl, about 10 U/μl, about 11 U/μl, about 12 U/μl, about 13 U/μl, about 14 U/μl, about 15 U/μl, about 16 U/μl, about 17 U/μl, about 18 U/μl, about 19 U/μl, about 20 U/μl, about 21 U/μl, about 22 U/μl, about 23 U/μl, about 24 U/μl, about 25 U/μl, about 26 U/μl, about 27 U/μl, about 28 U/μl, about 29 U/μl, about 30 U/μl, about 31 U/μl, about 32 U/μl, about 33 U/μl, about 34 U/μl, or about 35 U/μl DNA polymerase (e.g. Bst DNA polymerase).

In some embodiments, the warm-start CRISPR reaction mixture further comprises a reverse transcriptase. In some embodiments, the warm-start CRISPR reaction mixture further comprises at least 0.1 U/μl of the reverse transcriptase. In some embodiments, the warm-start CRISPR reaction mixture further comprises at least about 0.1 U/μl, at least about 0.2 U/μl, at least about 0.5 U/μl, at least about 1 U/μl, at least about 1.5 U/μl, at least about 2 U/μl, at least about 2.5 U/μl, or at least about 3 U/μl reverse transcriptase.

In some embodiments the CRISPR-reaction mixture additionally comprises a reporter molecule. In some embodiments, the reporter molecule generates a signal, wherein the signal is detected to determine the presence and/or amount of target within the sample. For example, in some embodiments the reporter molecule comprises a fluorescent moiety conjugated to a quencher. In an intact (e.g. uncleaved) state, the fluorescence from the fluorescent moiety is quenched, such as by resonance energy transfer. However, after cleavage of the reporter molecule, the fluorescent moiety is released from the quencher and the fluorescent signal becomes detectable. In some embodiments, the reporter molecule comprises a single stranded DNA reporter. In some embodiments, the reporter molecule comprises a single-stranded, non-target DNA sequence labeled with a fluorescent label. For example, the reporter molecule may comprise a single-stranded, non-target DNA sequence labeled with fluorescein or derivatives thereof (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)). The label may be present at the 5′ end or the 3′ end of the non-target DNA sequence. In some embodiments, the non-target DNA sequence comprises 2-10 amino acids. For example, the non-target DNA sequence may comprise 2-10 cysteine residues. For example, the non-target DNA sequence may comprise five cysteine residues (CCCCC; SEQ ID NO: 18).

In some embodiments, the endonuclease indiscriminately cleaves reporter molecules during cleavage of the amplicon generated as a result of isothermal amplification. Accordingly, binding of the Cas-crRNA complex (e.g. cas12a-crRNA complex) to the target amplicon induces cleavage of the reporter molecule. In contrast, in the absence of the target amplicon the endonuclease (e.g. cas12a) does not cause substantial (e.g. detectable) cleavage of the reporter molecule.

The warm-start CRISPR reaction mixture may further comprise additional reagents. For example, the warm-start CRISPR reaction mixture may comprise additional reagents to stabilize the sample (e.g. preservatives, inhibitors, etc.), along with suitable buffers, salts, dNTPs, and the like required for isothermal amplification and detection of the target, if present in the sample.

In some embodiments, the method comprises partitioning the warm-start CRISPR reaction mixture into a plurality of microwells. In some embodiments, the warm-start CRISPR reaction mixture has been contacted with the sample prior to partitioning. In other embodiments, the sample is present within the microwells. The method further comprises amplifying the target, if present in the sample, by isothermal amplification. In some embodiments, isothermal amplification comprises incubating the warm-start CRISPR reaction mixture at a temperature of about 50° C.-60° C. for at least about 10 minutes. For example, isothermal amplification may comprise incubating at a temperature of about 50° C.-60° C. for at least about 30 minutes, at least about 40 minutes, at least about 50 minutes, at least about 60 minutes, at least about 70 minutes, at least about 80 minutes, or at least about 90 minutes. In some embodiments, isothermal amplification comprises incubating at a temperature of about 50° C.-60° C. for about 90 minutes. In some embodiments, the temperature is about 50° C., 51° C., 52° C., 53° C., 54° C., 55° C., 56° C., 57° C., 58° C., 59° C., or 60° C. In some embodiments, the temperature is about 52° C.

In some embodiments, the method comprises signal in each of the plurality of microwells. For example, as described above incubation of the warm-start CRISPR reaction mixture following contact with the sample results in amplification of the target, if present in the sample. Using the primers described herein, an amplicon is generated that is bound by the endonuclease-crRNA complex (e.g. the Cas12a-crRNA complex). Binding of the complex allows for the endonuclease (e.g. cas12a) to also indiscriminately cleave the reporter molecule, thus generating a detectable signal (e.g. a detectable fluorescent signal). Detection of the signal in a well indicates the presence of the target in the well. In some embodiments, the number of detectable microwells can be quantified and used to determine the amount (e.g. level) of target in the sample.

The sample may be any suitable sample. In some embodiments, the sample is a biological sample (e.g. a sample obtained from a subject). The biological sample may comprise a fluid sample or a tissue sample. In some embodiments, the biological sample is a blood sample or a blood product such as serum or plasma. In some embodiments, the sample comprises urine. In embodiments, the sample is a respiratory specimen, including a nasal sample, an oropharyngeal sample, a mid-turbinate sample, sputum, endotracheal aspirate or bronchoalveolar lavage. In some embodiments, the sample is a cerebrospinal fluid sample. In some embodiments, the sample is a saliva sample. In some embodiments, the sample is a tissue sample. In some embodiments, the sample is obtained from a subject suspected of having a SARS-CoV-2 infection. In some embodiments, the subject is a human. The sample can be used directly as obtained from a patient or can be pre-treated, such as by heating, filtration, distillation, extraction, concentration, centrifugation, inactivation of interfering components, addition of reagents, and the like.

### 4. Kits

The disclosure further provides kits. In some embodiments, provided herein is a kit comprising one or more primers, compositions, or warm-start CRISPR reaction mixtures described herein. In some embodiments, provided herein is a kit comprising a primer having at least 90% sequence identity to SEQ ID NO: 1, a primer having at least 90% sequence identity to SEQ ID NO: 2, a primer having at least 90% sequence identity to SEQ ID NO: 4, a primer having at least 90% sequence identity to SEQ ID NO: 6, a primer having at least 90% sequence identity to SEQ ID NO: 7, and a primer having at least 90% sequence identity to SEQ ID NO: 8. In some embodiments, the kit comprises a primer having the amino acid sequence set forth in SEQ ID NO: 1, a primer having the amino acid sequence set forth in SEQ ID NO: 2, a primer having the amino acid sequence set forth in SEQ ID NO: 4, a primer having the amino acid sequence set forth in SEQ ID NO: 6, a primer having the amino acid sequence set forth in SEQ ID NO: 7, and a primer having the amino acid sequence set forth in SEQ ID NO: 8.

In some embodiments, the kit further comprises additional reagents useful for isothermal amplification of nucleic acid. For example, the kit may further comprise a DNA polymerase, a reverse transcriptase, pyrophosphatase, dNTPs, buffers, salts, stabilizers, etc.

In some embodiments, the kit further comprises reagents for CRISPR-based detection of the isothermally amplified nucleic acid. For example, the kit may further comprise a reporter molecule. In some embodiments, the reporter molecule comprises a single-stranded, non-target DNA sequence labeled with a fluorescent label. For example, the reporter molecule may comprise a single-stranded, non-target DNA sequence labeled with fluorescein or derivatives thereof (e.g., 5-fluorescein, 6-carboxyfluorescein, 3′6-carboxyfluorescein, 5(6)-carboxyfluorescein, 6-hexachloro-fluorescein, 6-tetrachlorofluorescein, fluorescein isothiocyanate, and the like)). The label may be present at the 5′ end or the 3′ end of the non-target DNA sequence. In some embodiments, the non-target DNA sequence comprises 2-10 amino acids. For example, the non-target DNA sequence may comprise 2-10 cysteine residues. For example, the non-target DNA sequence may comprise five cysteine residues (CCCCC (SEQ ID NO: 18)). In some embodiments, the kit further comprises an endonuclease (e.g. Cas12a). In some embodiments, the kit further comprises a target-specific cr-RNA. In some embodiments, the target-specific crRNA comprises a nucleotide sequence having at least 90% sequence identity to SEQ ID NO: 9. For example, in some embodiments the target-specific crRNA comprises a nucleotide sequence having at least 90%, at least 91%, at least 92%, at least 93%, at least 94%, at least 95%, at least 96%, at least 97%, at least 98%, at least 99%, or 100% sequence identity to SEQ ID NO: 9. In some embodiments, the target-specific crRNA comprises the nucleotide sequence of SEQ ID NO: 9.

The kit may be used for methods of detecting a target in a sample. In some embodiments, the target is nucleic acid. In some embodiments, the target is viral nucleic acid. For example, the target may be nucleic acid from an upper respiratory pathogen. For example, the kit may be used in methods of detecting SARS-CoV-2 nucleic acid in a sample. The kit may comprise additional components, including tubes (e.g. sample collection tubes, storage tubes, reaction tubes), buffers, stabilizers, salts, controls, calibrators, and the like. The kit may additionally comprise instructions for use. Instructions included in kits can be affixed to packaging material or can be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1

Presented herein is a digital warm-start CRISPR (WS-CRISPR) assay for sensitive quantitative detection of SARS-CoV-2 in clinical COVID-19 samples. The digital WS-CRISPR assay was established through partitioning a one-pot WS-CRISPR reaction into sub-nanoliter aliquots using commercial QuantStudio 3D digital chips. This reaction combined the reverse transcription dual-priming isothermal amplification (RT-DAMP) (23) and CRISPR-Cas12a-based fluorescence detection in one-pot format and is efficiently initiated at 52° C., the firstly reported digital warm-start CRISPR-Cas12a assay. Thus, the digital WS-CRISPR thoroughly addresses premature target amplifications at room temperature. Through targeting the SARS-CoV-2's nucleoprotein (N) gene, DWS-CRISPR is able to detect down to 50 copies/μl SARS-CoV-2 RNA in the sample, showing high sensitivity. In addition, digital WS-CRISPR is validated by quantitatively determining 32 clinical swab samples and 3 clinical saliva samples. More importantly, digital WS-CRISPR can directly detect SARS-CoV-2 in heated saliva samples without RNA extraction. This digital WS-CRISPR method is therefore a reliable, sensitive and straightforward CRISPR assay which facilitates SARS-CoV-2 detection toward digitized quantification.

### Results

### Overview of Digital Warm-Start CRISPR Assay

As shown in FIG. 1a, one-pot warm-start CRISPR reaction mixture is first prepared in one tube, containing Cas12a-crRNA complex, six DAMP primers (two outer primers of FO and RO, two inner primers of FI and RI, and two competition primers of FI and RI), nontarget single-stranded DNA fluorophore-quencher (ssDNA-FQ) reporter, SuperScript IV reverse transcriptase, Bst DNA polymerase, pyrophosphatase (PPase) and SARS-CoV-2 RNA target. To achieve digital detection, the prepared reaction mixture is distributed into a QuantStudio 3D digital chip (Thermo Fisher Scientific). This chip is etched with 20,000 consistently-sized hexagon wells (max width of 60 m) in a silicon substrate and can partition the mixture into over ten thousand sub-nanoliter (˜0.7 nL) microreactions. After 90 min incubation at 52° C., the microreaction with target RNA indicates strong green fluorescence (positive spots), whereas not in those without targets (negative spots). The target RNA can be quantified through testing and counting the number of positive spots of the chips.

The WS-CRISPR assay combines RT-DAMP amplification with CRISPR-Cas12a detection in one-pot. The DAMP amplification is one variant of loop-mediated isothermal amplification using a new primer design strategy (23). Each inner primer of the DAMP amplification is formed with two target sites with a distance of below 40 nt and a pair of competition primers is added to mediate pair-priming strand extension (FIG. 1b). The DAMP/RT-DAMP amplification has improved the detection sensitivity and also generated ultralow nonspecific signals (23). In the digital WS-CRISPR assay, six DAMP primers recognize six distinct sites in target sequences to initiate self-priming and pair-priming (dual-priming) isothermal amplification, producing multiple amplicons with closed loop structures. Simultaneously, Cas12a-cRNA complex specifically binds the sites in these amplicons to activate Cas12a's collateral cleavage activity, thereby indiscriminately cleaving surrounding nontarget ssDNA-FQ reporter to generate increased fluorescence. The ssDNA-FQ is a 5-cytosin nucleotide single-stranded DNA (5′-CCCCC-3′) labeled with FAM (Fluorescein) at 5′ end and Iowa Black FQ quencher at 3′ end, due to its higher affinity to Cas12a (24). Fluorescence is quenched via resonance energy transfer in intact ssDNA-FQ reporters, but can be recovered after activated Cas12a cleaves the reporters. This digital WS-CRISPR method is a warm-start assay and thoroughly circumvents the influence of premature target amplification. This strategy is very beneficial for handling massive quantitation tests and requires only a short preparation time (e.g., typically 5-10 min preparation per chip) at room temperature.

### Overcoming Challenges for the WS-CRISPR Assay

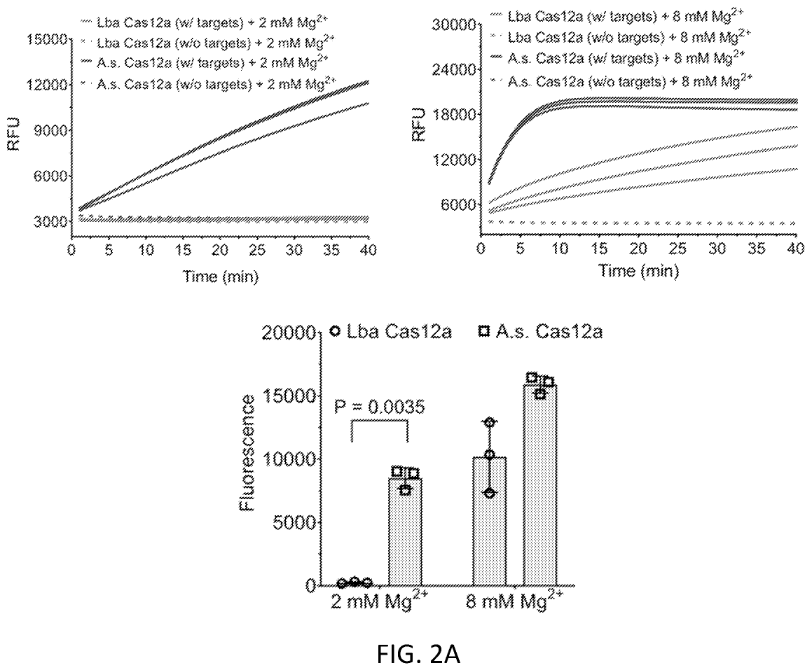

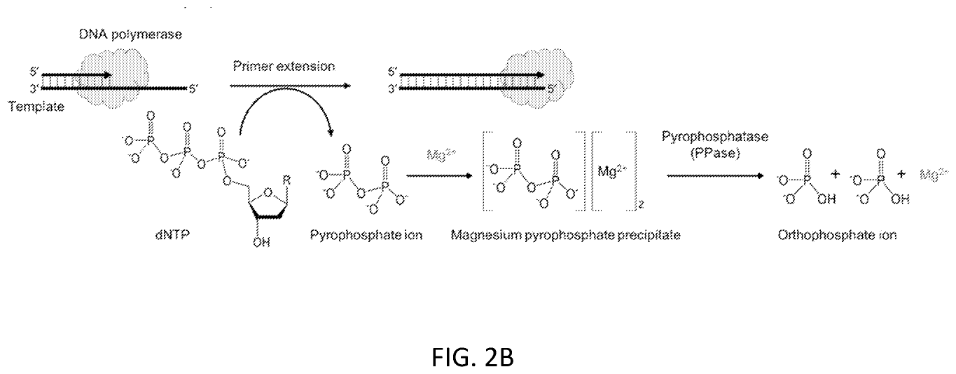

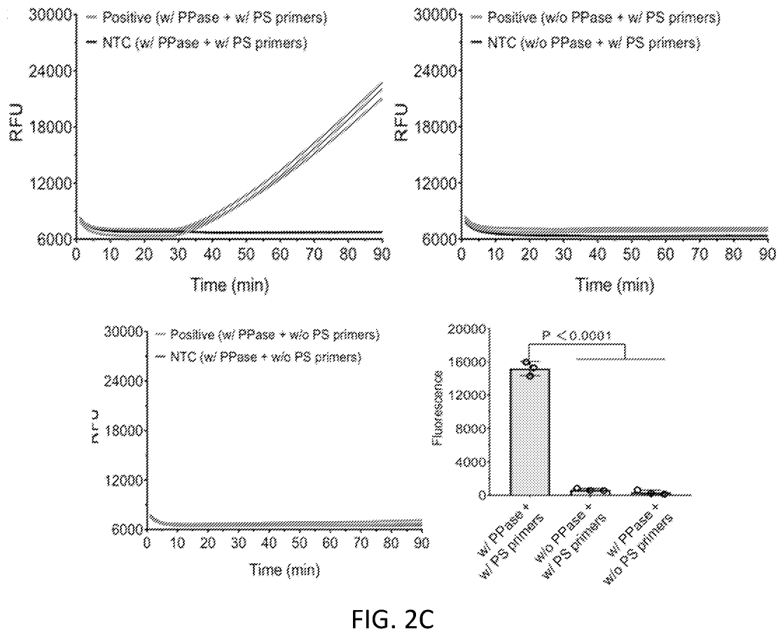

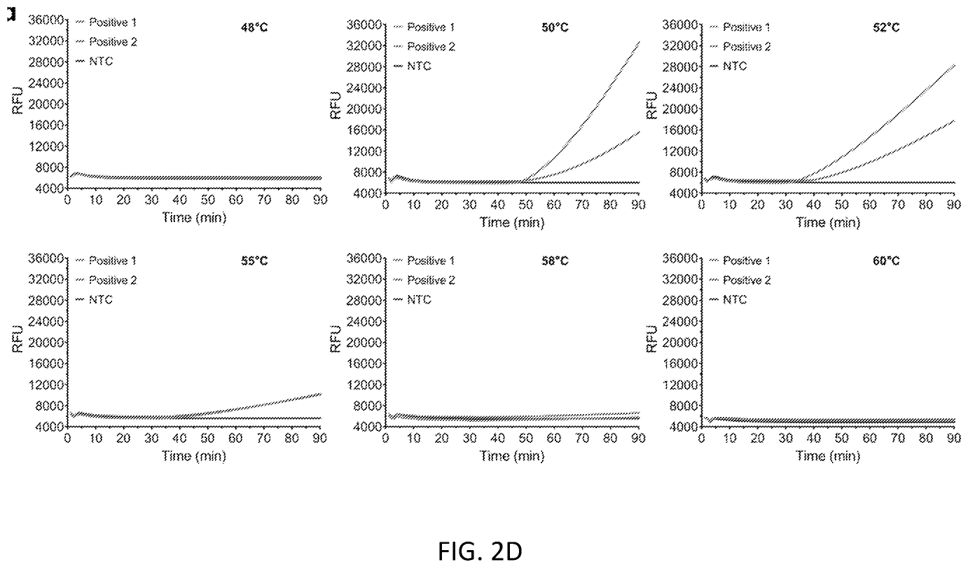

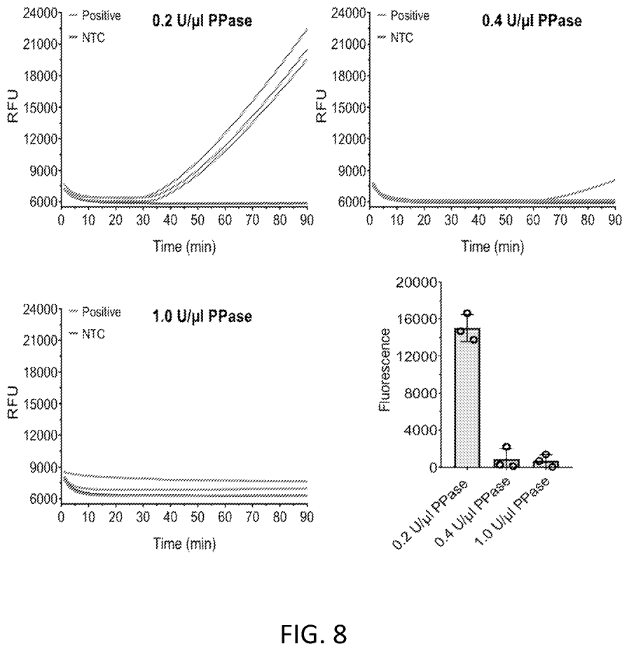

There remains a challenge to directly couple LAMP or DAMP amplification with CRISPR-Cas12a detection in a “one-pot” format due to the significant difference in their reaction buffer compositions and reaction temperatures. For example, one of the major concerns is the concentration of Mg²⁺. The cleavage of Cas12a nucleases for both on-target and collateral activity is typically high-Mg²⁺-dependent (25-27), while not for LAMP or DAMP amplification. To enable high sensitive nucleic acid detection, different Cas12a nucleases were evaluated and compared evaluated at various concentration of Mg²⁺. As shown FIG. 2a, when reducing the Mg²⁺ concentration from 8 mM to 2 mM, the detection efficiency of the CRISPR-Cas12a dramatically decreases. Interestingly, the Cas12a from Recombinant Acidaminococcus sp. BV3L6 (A.s. Cas12a) still has detectable collateral cleavage activity at 2 mM Mg²⁺ Whereas, 2 mM Mg²⁺ completely inhibits the activity of Cas12a from Lachnospiraceae bacterium ND2006 (Lba Cas12a). Thus, A.s. Cas12a was used in the WS-CRISPR assay due to its tolerance to low Mg²⁺ ion concentrations. In addition, during isothermal amplification, primer extension by DNA polymerase continually consumes dNTPs and produces a large number of pyrophosphate ions that can chelate Mg²⁺ to form insoluble magnesium pyrophosphate precipitate as the byproduct of reaction (FIG. 2b), thereby consuming a large amount of free Mg²⁺ and significantly weakening the collateral cleavage activity of CRISPR-Cas12a nuclease. To this end, pyrophosphatase (PPase) was added into the WS-CRISPR reaction system to degrade the magnesium pyrophosphate precipitate and release the free Mg²⁺ As shown in FIG. 8, the optimal concentration of PPase is 0.2 U/μl in the WS-CRISPR assay.

Most Cas12a nucleases have an optimal activity at 37° C., but LAMP/DAMP amplification powered by Bst DNA polymerase typically requires high temperature of 60-65° C. (23, 28). To develop a “one-pot” assay, phosphorothioated inner primers of FI an RI (Table 1) were employed in the WS-CRISPR assay to reduce the reaction temperature of isothermal amplification.

TABLE 1
The list of all used sequences in this study

| Item | Sequence (5′-3′) |
|---|---|
| DAMP forward outer | GGCTTCTACGCAGAAGGGA |
| primer (FO) targeting | (SEQ ID NO: 1) |
| SARS-CoV-2 N gene | |
| DAMP reverse outer | AATCTGTCAAGCAGCAGCA |
| primer (RO) targeting | (SEQ ID NO: 2) |
| SARS-CoV-2 N gene | |
| DAMP forward inner | TTGAACTGTTGCGACTACG |
| primer (FI) targeting | TGTTTTAGCCTCTTCTCGT |
| SARS-CoV-2 N gene | TCCTCAT |
| | (SEQ ID NO: 3) |
| Phosphorothioated FI | T*T*G*A*A*C*T*G*T*T* |
| (PS-FI) | G*C*G*A*C*T*A*C*G*T* |
| | G*TTTTAGCCTCTTCTCGT |
| | TCCTCAT |
| | (SEQ ID NO: 4) |
| DAMP reverse inner | GAACTTCTCCTGCTAGAAT |
| primer (RI) targeting | GGCTTTTGCAAGAGCAGCA |
| SARS-CoV-2 N gene | TCACCG |
| | (SEQ ID NO: 5) |
| Phosphorothioated RI | G*A*A*C*T*T*C*T*C*C* |
| (PS-RI) | T*G*C*T*A*G*A*A*T*G* |
| | G*C*TTTTGCAAGAGCAGC |
| | ATCACCG |
| | (SEQ ID NO: 6) |
| DAMP forward | TTGAACTGTTGCGACTACG |
| competition primer | TG (SEQ ID NO: 7) |
| (FC) targeting | |
| SARS-CoV-2 N gene | |
| DAMP reverse | GAACTTCTCCTGCTAGAAT |
| competition primer | GGC (SEQ ID NO: 8) |
| (RC) targeting | |
| SARS-CoV-2 N gene | |
| DAMP-crRNA targeting | UAAUUUCUACUAAGUGUAG |
| SARS-CoV-2 N gene | AUCCCUACUGCUGCCUGGA |
| | GUUGAA |
| | (SEQ ID NO: 9) |
| Synthetic SARS-CoV-2 | GCCATTCGAGCAGGAGAAT |
| N DNA fragment | TTCCCCTACTGCTGCCAGG |
| | AGTTGAATTTCTTGAATTA |
| | CCGCGACTACGTG |
| | (SEQ ID NO: 10) |
| nCOV_N1 RT-qPCR | GACCCCAAAATCAGCGAAA |
| forward primer (1) | T (SEQ ID NO: 11) |
| nCOV_N1 RT-qPCR | TCTGGTTACTGCCAGTTGA |
| reverse primer (1) | ATCTG |
| | (SEQ ID NO: 12) |
| nCOV_N1 RT-qPCR | FAM-ACCCCGCATTACGTT |
| probe (1) | TGGTGGACC-BHQ1 |
| | (SEQ ID NO: 13) |
| AIOD-CRISPR forward | AGGCAGCAGTAGGGGAACT |
| primer (FP) | TCTCCTGCTAGAAT |
| targeting SARS-CoV-2 | (SEQ ID NO: 14) |
| N gene (2) | |
| AIOD-CRISPR reverse | TTGGCCTTTACCAGACATT |
| primer (FP) | TTGCTCTCAAGCTG |
| targeting SARS-CoV-2 | (SEQ ID NO: 15) |
| N gene (2) | |
| AIOD-CRISPR forward | UAAUUUCUACUAAGUGUAG |
| crRNA (crRNA1) | AUCAUCACCGCCAUUGCCA |
| targeting SARS-CoV-2 | GCC (SEQ ID NO: 16) |
| N gene (2) | |
| AIOD-CRISPR reverse | UAAUUUCUACUAAGUGUAG |
| crRNA (crRNA2) | AUUUGCUGCUGCUUGACAG |
| targeting SARS-CoV-2 | AUU (SEQ ID NO: 17) |
| N gene (2) | |

* The nucleotides with phosphorothioate modifications.

Therefore, at least in part through supplementing PPase and employing phosphorothioated inner primers, one-pot isothermal WS-CRISPR assay was successfully developed (FIG. 2c).

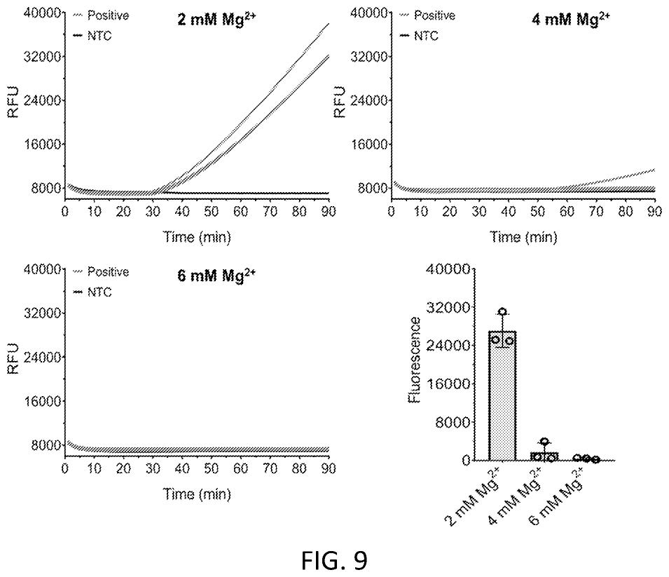

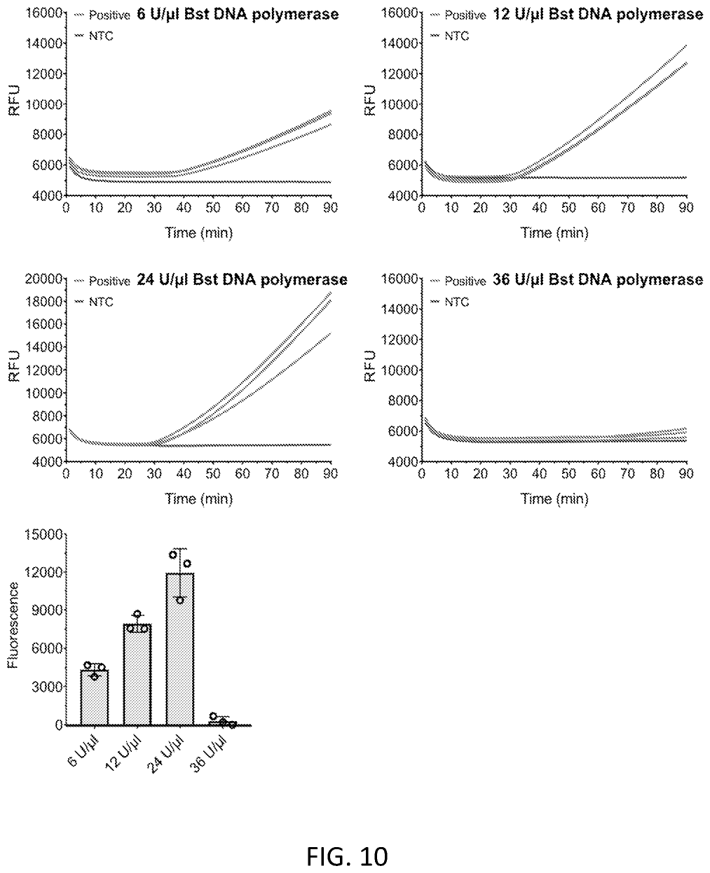

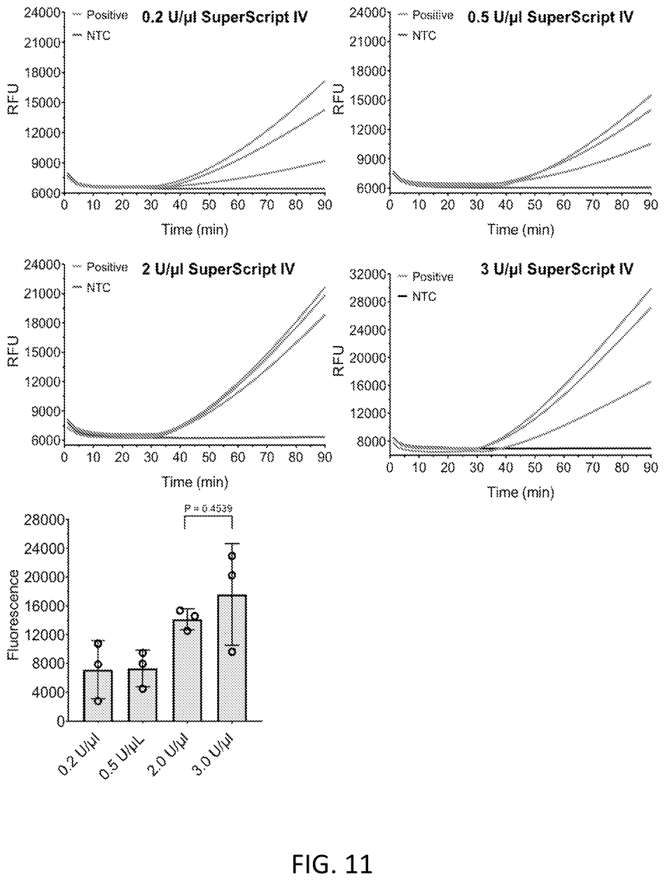

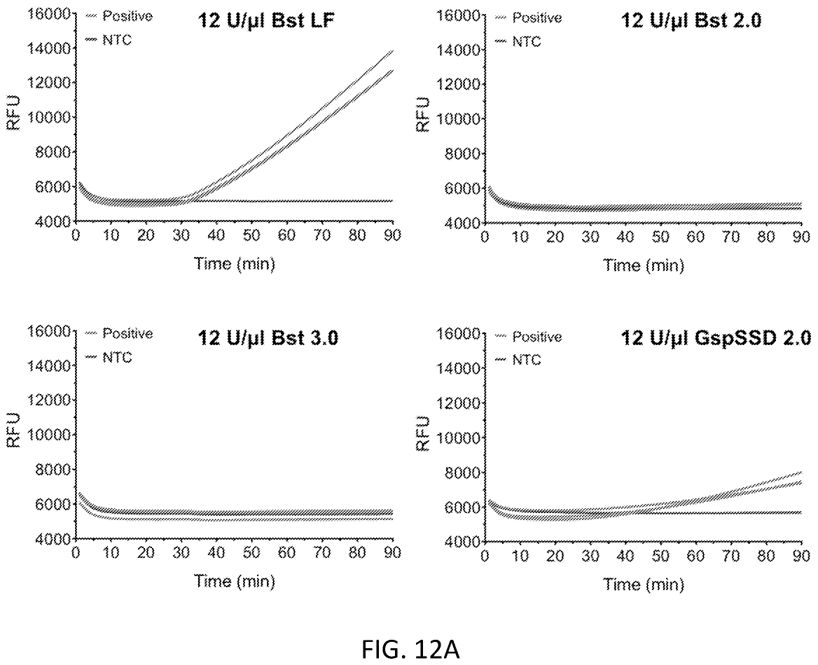

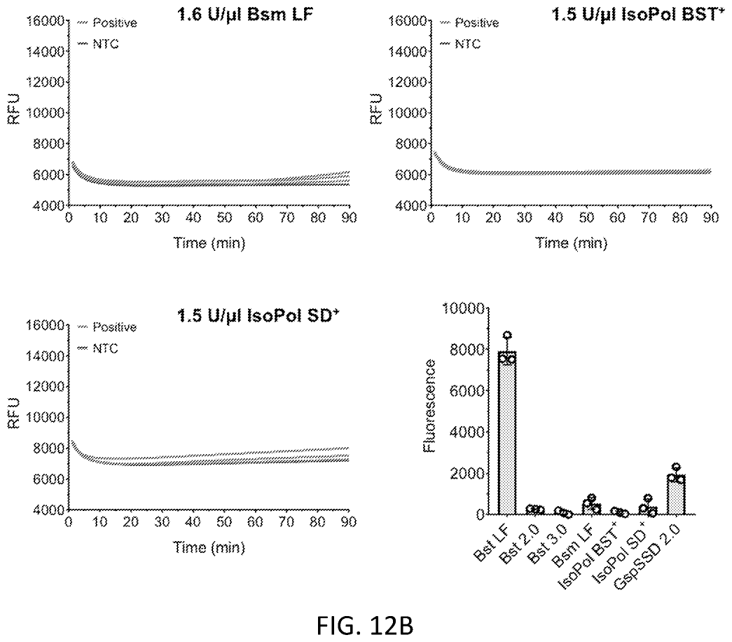

Concentrations of PPase, Mg²⁺ ion, Bst DNA polymerase, and SuperScript IV reverse transcriptase were further evaluated. As shown in FIGS. 8-11, the optimal concentrations were determined as 0.2 U/μl PPase (FIG. 8), 2 mM Mg²⁺ (FIG. 9), 24 U/μl Bst DNA polymerase (FIG. 10), and 2 U/μl SuperScript IV (FIG. 11). In addition, several different DNA polymerases were investigated and compared, including Bst DNA polymerase (large fragment), Bst 2.0 DNA polymerase, Bst 3.0 DNA polymerase, GspSSD 2.0 DNA polymerase, Bsm DNA polymerase (large fragment), IsoPol BST⁺ DNA polymerase, and IsoPol SD⁺ DNA polymerase. As shown in FIG. 12, the best DNA polymerase for the WS-CRISPR is Bst DNA polymerase (large fragment). Furthermore, the effect of reaction temperatures from 48° C. to 60° C. on the one-pot WS-CRISPR assay was assessed. FIG. 2d shows that the optimal temperature of the WS-CRISPR assay is 52° C. and the reaction cannot be initiated at 48° C., demonstrating that the one-pot isothermal CRISPR method is a warm-start assay which is initiated at 50° C. or so.

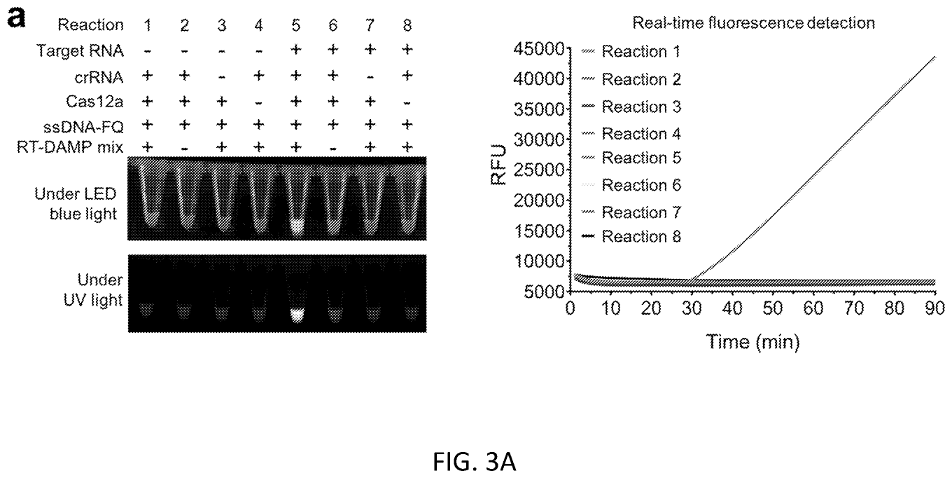

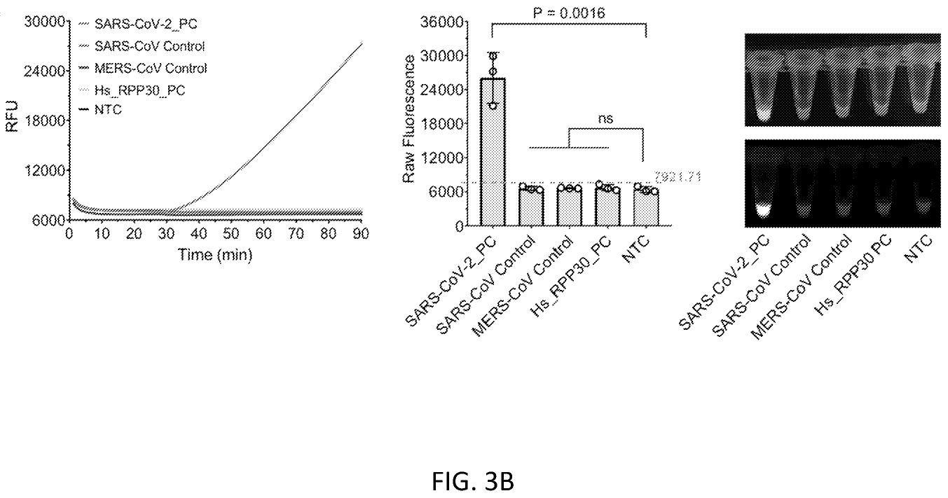

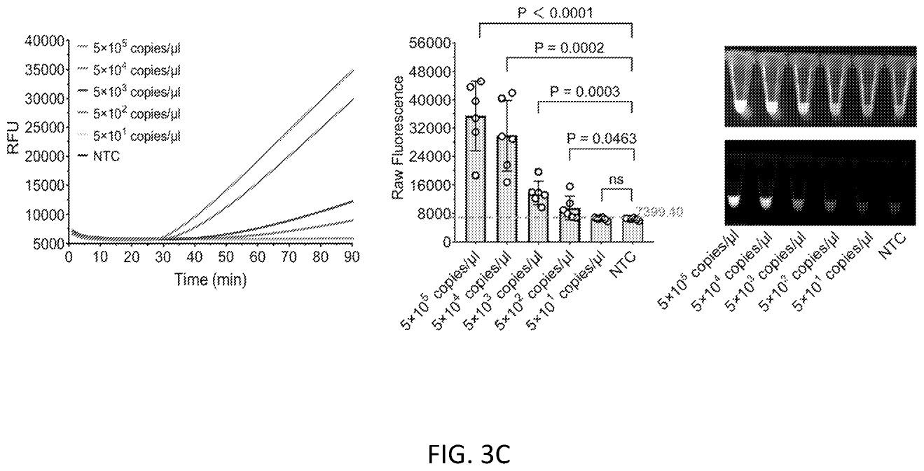

As shown in FIG. 3a, the WS-CRISPR reaction is only initiated when all the components of the RT-DAMP and CRISPR-Cas12a are mixed in the “one-pot” reaction solution (FIG. 1). FIG. 3b shows that the WS-CRISPR assay has a high specificity to detect SARS-CoV-2. By detecting various concentrations of SARS-CoV-2 RNA, the WS-CRISPR assay is able to detect down to 500 copies/μl SARS-CoV-2 RNA (50 copies/μl RNA in the reaction) within 90 min (FIG. 3c), showing a high detection sensitivity. Furthermore, FIG. 3 demonstrates that the detection results of the WS-CRISPR assay can be visually read out based on the fluorescence imaging of reaction tubes under LED blue light and UV light. In sum, described herein is a one-pot warm-start CRISPR assay having high sensitivity and specificity for SARS-CoV-2 detection in both real-time fluorescence monitoring and endpoint visual readout. The WS-CRISPR assay is typically initiated at above 50° C., presenting the first report of one-pot warm-start CRISPR-Cas12a assay.

### Development of Digital WS-CRISPR Assay

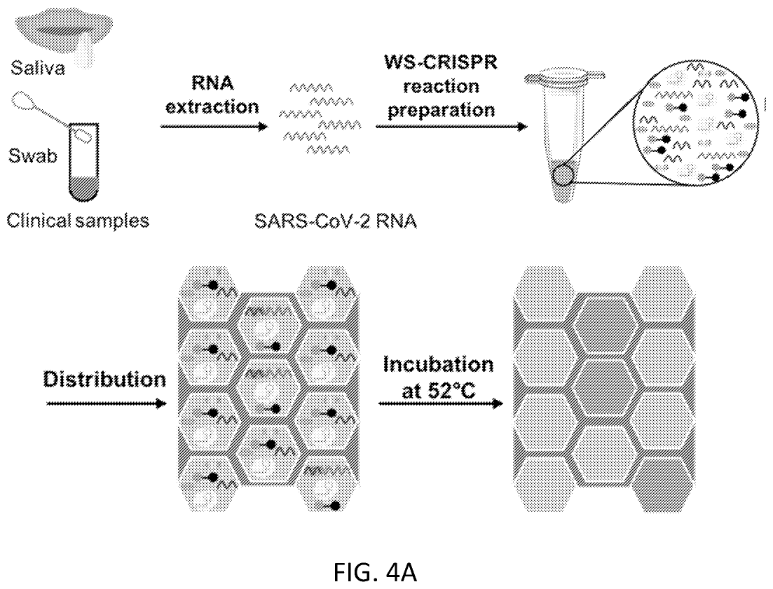

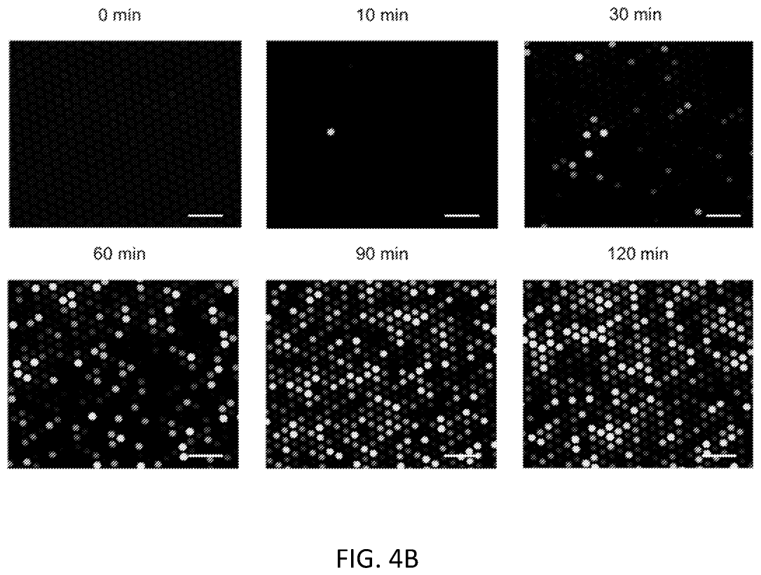

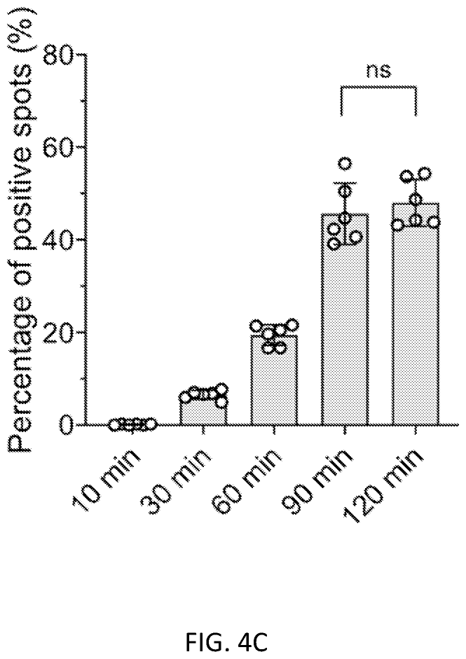

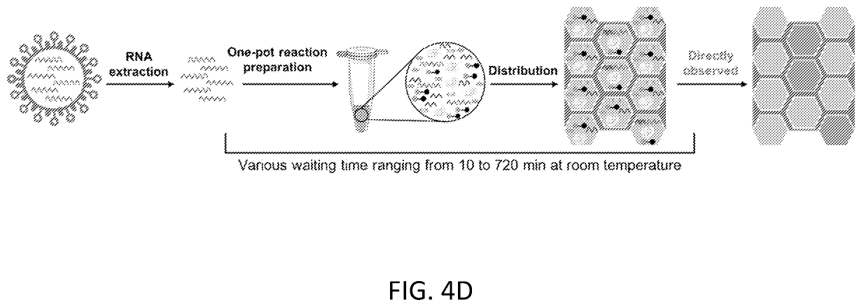

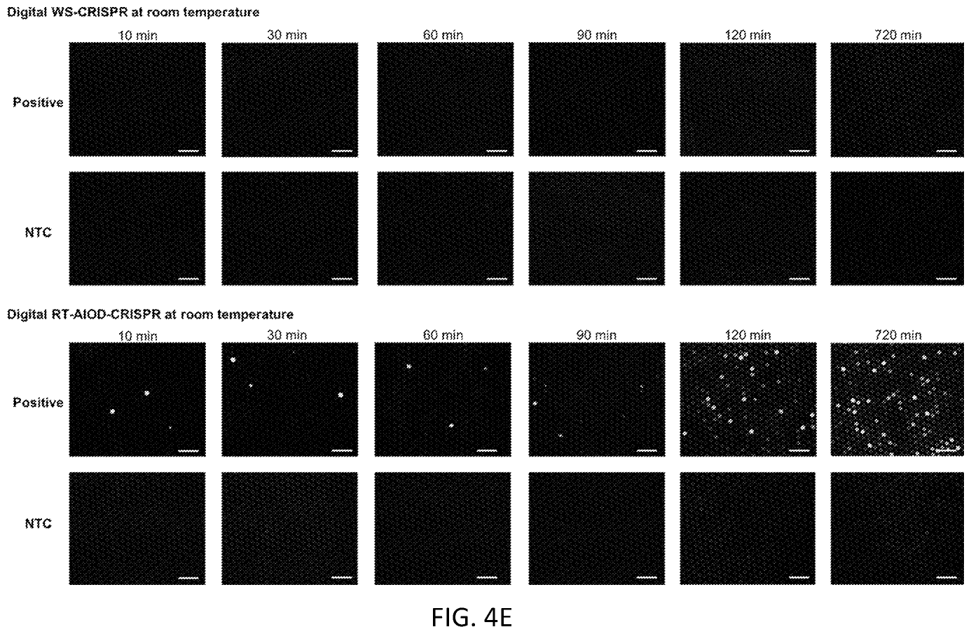

The digital WS-CRISPR assay is developed through partitioning the newly established one-pot WS-CRISPR reaction mixture into sub-nanoliter microreactions in the QuantStudio 3D digital chips. As shown in FIG. 4a, a typical workflow for digital WS-CRISPR assay consists of RNA extraction from clinical samples, one-pot reaction mixture preparation, distribution of the reaction mixture into the chip, and incubation at 52° C. First, digital WS-CRISPR assays with various incubation times (e.g., 10, 30, 60, 90 and 120 min) were investigated. FIGS. 4b and 4c shows that a 90-min incubation is enough for the digital WS-CRISPR assay to reach the max percentage of positive spots.

Next, the effect of waiting time at room temperature on digital CRISPR assays was evaluated. Typically, it takes 5-10 min to prepare one-pot CRISPR reaction mixture. Various waiting times at room temperature during the one-pot reaction preparation and distribution steps were tested (FIG. 4D) and compared to reported RT-AIOD-CRISPR assays (19). As shown in FIG. 4E, digital RT-AIOD-CRISPR assays has obvious premature target amplification to present positive spots as short as 10 min at room temperature, which is consistent with data for one-pot RT-RPA-based CRISPR reactions (21). In contrast, the digital WS-CRISPR assay described herein is stable without any observed positive spots even after 720-min waiting time at room temperature. Thus, this digital WS-CRISPR assay is superior and enables a reliable and user-friendly detection.

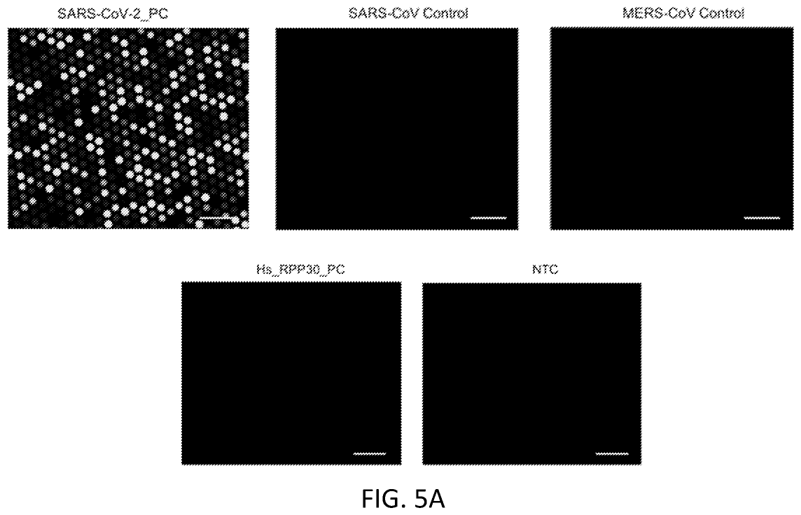

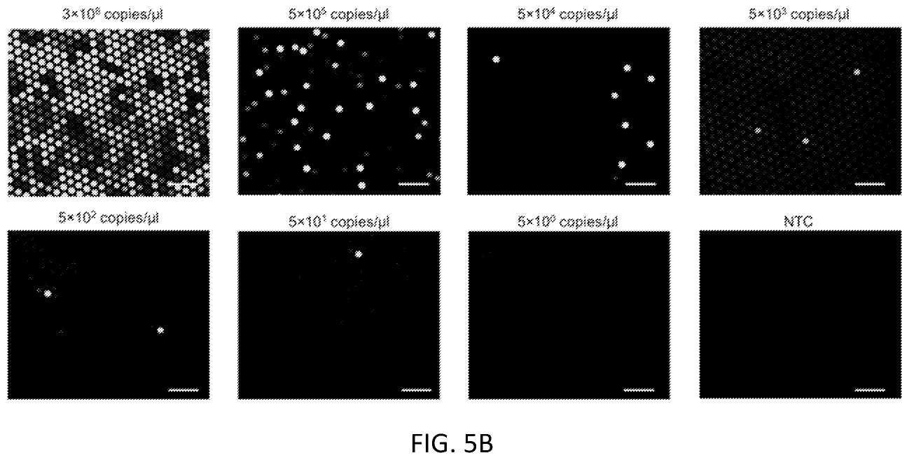

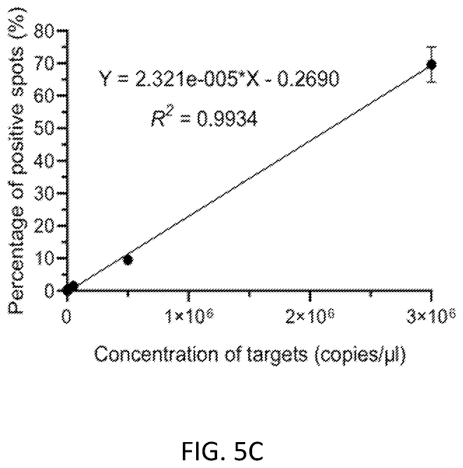

The specificity of the digital WS-CRISPR was tested using non-SARS-CoV-2 nucleic acids. As shown in FIG. 5a, positive spots are observed in the chip with the SARS-CoV-2 positive control loaded, and are not observed for other non-target nucleic acids, thus demonstrating that the digital WS-CRISPR possesses high specificity for SARS-CoV-2 detection. By testing various concentrations of SARS-CoV-2 RNA, the detection sensitivity was also investigated. As showed in FIG. 5b, digital WS-CRISPR is able to detect down to 50 copies/μl SARS-CoV-2 RNA (equivalently 5 copies/μl RNA molecules in the chip), a 10-fold improvement compared with the bulk reaction assay in the tube format (FIG. 3c). FIG. 5b also indicates a linear relationship between the concentration of targets (from 5×10³ and 3×10⁶ copies/μl) and the percentage of positive spots. Unlike tube-based quantitation detection, digital detection has the advantage of absolutely quantifying target without a standard curve (30).

### Clinical Validation of Digital WS-CRISPR Assay

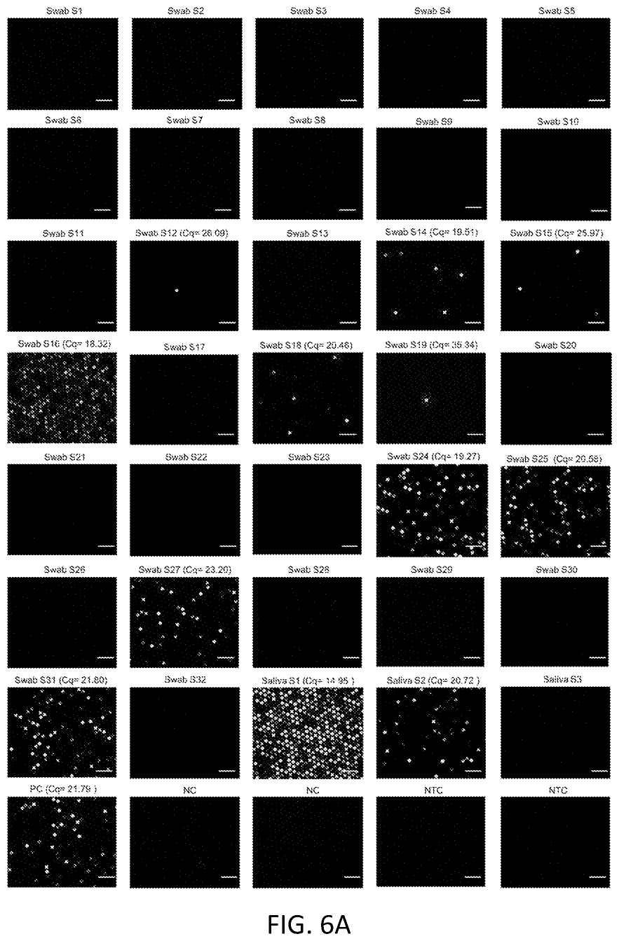

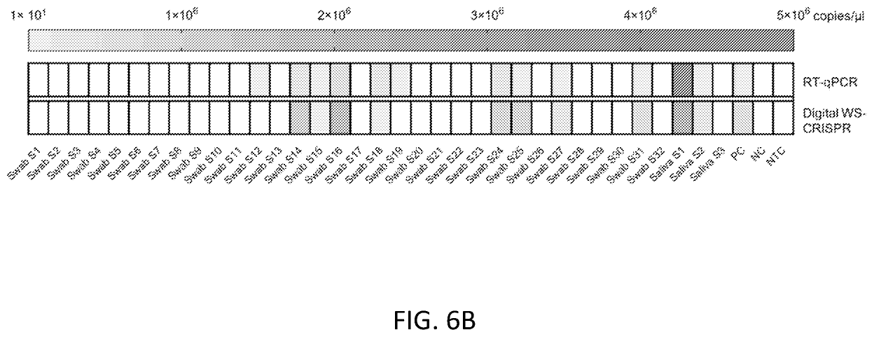

In order to demonstrate the feasibility for clinical SARS-CoV-2 sample testing, the digital WS-CRISPR assay was used to detect SARS-CoV-2 RNA extracted from 32 clinical swab samples and 3 clinical saliva samples. Meanwhile, an in-home RT-qPCR assay using the U.S. CDC-approved SARS-CoV-2 N1 gene's primers and probes (provided by Integrated DNA Technologies) were set up as the parallel experiment. As shown in FIG. 6a, 12 positive samples including the positive control were consistently detected and identified by digital WS-CRISPR assays, while all negative samples showed negative signals, showing a 100% agreement with that of RT-PCR method. FIG. 6b shows the determined concentrations of SARS-CoV-2 RNA in the 12 positive samples and the positive control by digital WS-CRISPR and RT-qPCR methods. The averaged viral loading was quantified with the range from 1.4×10⁴ to 2.3×10⁶ copies/μl, showing similar order of magnitude as those determine by RT-qPCR. Accordingly, the digital WS-CRISPR assay is able to quantify the SARS-CoV-2 RNA extracted from both clinical swab and saliva samples, showing a comparable performance with conventional RT-qPCR method. In addition, the digital WS-CRISPR is the first digital CRISPR assay validated using clinical samples.

### Direct SARS-CoV-2 Detection in Saliva Samples by Digital WS-CRISPR Assay

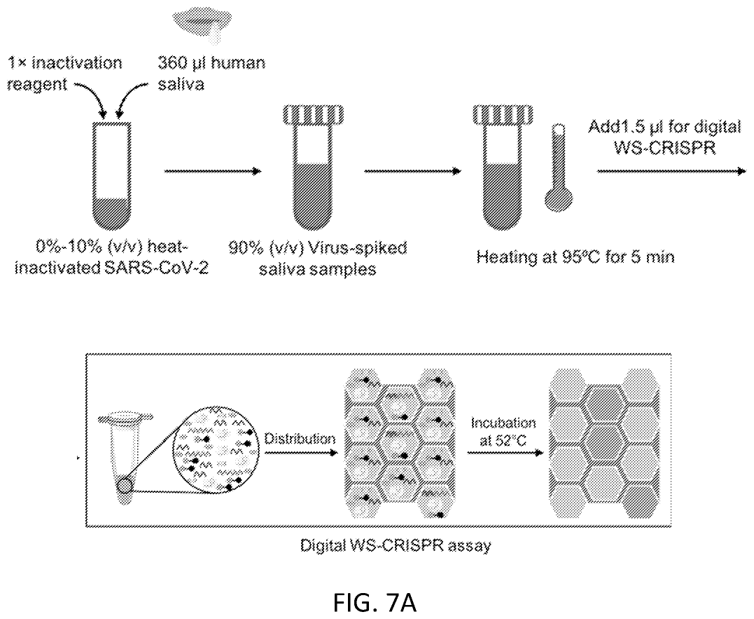

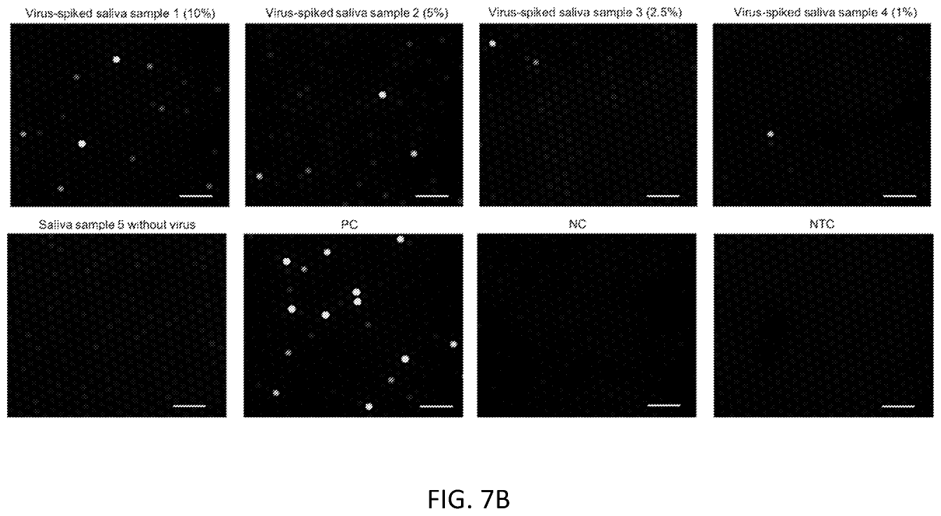

Saliva testing is advantageous over swab testing, since saliva samples can be self-collected by patients themselves, avoiding direct interaction between health care workers and patients (32). Given this, whether the digital WS-CRISPR assay can directly detect SARS-CoV-2 in crude saliva samples without nucleic acid extraction step was tested. As shown in FIG. 7a, each saliva sample contained 90% (v/v) of human saliva, 0%-10% (v/v) of spiked heat-inactivated SARS-CoV-2 from BEI Resources (Catalog #NR-52350), and 1× inactivation reagent developed by Rabe and Cepko (7). After heating at 95° C. for 5 min, 1.5 μl of the saliva samples was directly added into the digital WS-CRISPR reaction system. As shown in FIG. 7b, the digital WS-CRISPR assay is successfully able to detect the SARS-CoV-2 spiked in the saliva samples without need for RNA extraction and purification, exhibiting high tolerance to potential inhibitors in saliva samples due to reaction partitioning. Thus, this interesting finding suggests that the digital WS-CRISPR assay described herein has the potential to directly detect SARS-CoV-2 from crude clinical saliva specimens through simple heating treatment, facilitating rapid and early molecular diagnostics of COVID-19 infection.

### CONCLUSIONS

Described herein is a digital warm-start CRISPR-Cas12a (WS-CRISPR) assay for sensitive quantitation of SARS-CoV-2 from clinical samples. A one-pot warm-start CRISPR reaction combing a reverse transcription isothermal nucleic acid amplification (RT-DAMP) and CRISPR-Cas12a-based detection was used. To coordinate these two functions in one solution, pyrophosphatase and phosphorothioated inner primers were used to keep free magnesium ions stable for efficient CRISPR-Cas12a-based detection and to mediate efficient reverse transcription isothermal amplification at unfavorable temperatures such as 52° C., respectively. Through partitioning this one-pot reaction mixture into sub-nanoliter microreactions using QuantStudio 3D digital chips, the warm-start digital WS-CRISPR assay was successfully developed.

Digital WS-CRISPR assay offers several remarkable advantages. First, digital WS-CRISPR enables the successful one-pot and one-step reaction with Bst DNA polymerase-based reverse transcription isothermal amplifications (e.g., RT-DAMP and RT-LAMP) and CRISPR-Cas12a detection system, not relying on CRISPR-Cas12b which needs a relatively long crRNA (33, 34). Second, digital WS-CRISPR assay is typically initiated at a warm temperature (e.g., −50° C.) without regard to premature target amplification, an uncontrollable challenge in current digital CRISPR assays. Third, digital WS-CRISPR has high detection specificity and 10-fold higher sensitivity than tube-based bulk assay format. By targeting the SARS-CoV-2's N gene, digital WS-CRISPR is able to quantify down to 50 copies/μl SARS-CoV-2 RNA in the sample, equivalently 5 copies/μl SARS-CoV-2 RNA in the chip. Fourth, digital WS-CRISPR can quantify the viral loading in SARS-CoV-2's clinical samples, benefiting assessing the COVID-19 infectivity and the efficacy of antiviral drugs. Last, the digital WS-CRISPR assay can be used for direct saliva SARS-CoV-2 testing without time-consuming RNA extraction. This potential feature not only facilitates the COVID-19 diagnosis but also lowers the risk of infection in health workers without directly sampling from patients. In sum, the digital WS-CRISPR assay described herein provides a reliable, sensitive and straightforward platform for SARS-CoV-2 quantitative detection.

### Methods

### Reagents and Samples

Bovine serum albumin (BSA, 20 mg/ml), EnGen Lba Cas12a (100 μM), deoxynucleotide (dNTP) mix (10 mM of each), RNase inhibitor (Murine, 40,000 U/ml), extreme thermostable single-stranded DNA binding protein (ET-SSB, 500 μg/ml), isothermal amplification buffer pack (10×; containing 200 mM Tris-HCl, 500 mM KCl, 100 mM (NH₄)₂SO₄, 20 mM MgSO₄, 1% Tween 20, and pH=8.8 at 25° C.), thermostable inorganic pyrophosphatase (PPase, 2,000 U/ml), Bst DNA polymerase (large fragment), Bst 2.0 DNA polymerase, Bst 3.0 DNA polymerase, and nuclease-free water were purchased from New England BioLabs (Ipswich, MA). GspSSD 2.0 DNA polymerase was from OptiGene (West Sussex, UK). IsoPol BST⁺ and IsoPol SD⁺ polymerases were purchased from ArcticZymes Technologies (Norway). Invertase from Saccharomyces cerevisiae (Grade VII, >300 U/mg), taurine (>99%, 10 g), TCEP-HCl (Reagent Grade, 5 g), and NaOH (>98%, 500 g) were purchased from Sigma-Aldrich (St. Louis, MO). UltraPure EDTA (0.5 M, pH=8), Bsm DNA Polymerase, large fragment (8 U/μl), SuperScript IV reverse transcriptase (200 U/μL), QuantStudio 3D digital PCR 20K chip kit (Version 2), and digital PCR master mix were purchased from Thermo Fisher Scientific (Waltham, MA). Normal saliva human fluid was purchased from MyBioSource (San Diego, CA). Heat-inactivated SARS-CoV-2 (Isolate USA-WA1/2020, NR-52350) was from BEI Resources (Manassas, VA). Synthetic SARS-CoV-2 RNA control (MN908947.3) with a coverage of greater than 99.9% of the bases of the SARS-CoV-2 viral genome was purchased from Twist Bioscience (San Francisco, CA). Alt-R A.s. Cas12a Ultra (500 μg; 64 μM), SARS-CoV-2 positive control (SARS-CoV-2_PC, Catalog #10006625), SARS-CoV control (Catalog #10006624), and Middle East respiratory syndrome coronavirus control (MERS-CoV control, Catalog #10006623), human RPP30 gene control (Hs_RPP30_PC, Catalog #10006626), nCOV_N1 Forward Primer Aliquot (50 nmol, Catalog #10006821), nCOV_N1 Reverse Primer Aliquot (50 nmol, Catalog #10006822), and nCOV_N1 Probe Aliquot (50 nmol, Catalog #10006823) were purchased from Integrated DNA Technologies (Coralville, IA). All clinical samples were handled in compliance with ethical regulations and the approval of Institutional Review Board of the University of Connecticut Health Center (protocol #: P61067).

Design of Primers and crRNA

Six DAMP primers and one CRISPR-Cas12a's crRNA were designed to target seven distinct sites in the 173 bp SARS-CoV-2 N gene fragment with the location from 28769 to 28941 in the viral genome (GenBank accession MW202218.1). The selected primer or crRNA recognition sites were checked to be highly conserved based on the GISAID-provided genomic epidemiology of hCoV-19 for 3564 genomes sampled between December 2019 and November 2020 (as of Nov. 11, 20202. https://www.gisaid.org/epiflu-applications/phylodynamics/). The DAMP primers can be manually designed using the OligoAnalyzer Tool (https://www.idtdna.com/pages) and the PrimerExplorer (http://primerexplorer.jp/e/) according to previously reported design principles (23). Also, they can be designed using the online DAMP primer design platform (https://github.com/xuzhiheng001/DAMP-Design). CRISPR-Cas12a's crRNA targeting the middle site of DAMP amplification region (see FIG. 1) was also designed using the OligoAnalyzer Tool and its sequence was checked by against MERS-CoV and SARS-CoV gene sequences. Primers, crRNA, ssDNA-FQ reporter, and SARS-CoV-2 N DNA fragment were synthesized from Integrated DNA Technologies (Coralville, IA). All the sequence information of used primers and crRNAs has been listed in Table 1.

### One-Pot Warm-Start CRISPR Assay

The one-pot isothermal CRISPR assay system was prepared separately as Component A and B. Component A consisted of 1× isothermal amplification buffer (20 mM Tris-HCl, 50 mM KCl, 10 mM (NH₄)₂SO₄, 2 mM MgSO₄, 0.1% (v/v) Tween 20, and pH=8.8 at 25° C.), 2 U/μl SuperScript IV reverse transcriptase, 50 mM taurine, 1 U/μl invertase, 0.01 mg/ml BSA, 0.4 mM each of dNTPs, 0.2 μM FO primer, 0.2 μM RO primer, 1.6 μM PS-FI primer, 1.6 μM PS-RI primer, 1.6 μM FC primer, 1.6 μM RC primer, 10 μM ssDNA-FQ, 20 ng/μl ET SSB, 24 U/μl Bst DNA polymerase (large fragment), 0.2 U/μl PPase, and 1 U/μl RNase inhibitor. Component B contained 1.28 μM A.s. Cas12a (Ultra) and 1.2 μM DAMP-crRNA (table 1). All the indicated concentrations were calculated based on the finally assembled reaction system. In a typical 15-1 assay, 12.5 μl Component A was first mixed with 1.5 μl of the target solution and then supplemented with 1.0 μl Component B. The assembled reaction mixture was then incubated at 52° C. for 90 min in the Bio-Rad CFX96 Touch Real-Time PCR Detection System (Hercules, CA) for real-time fluorescence detection. After incubation, the tubes were placed in the Maestrogen UltraSlim LED blue light illuminator (Pittsburgh, PA) or the Bio-Rad ChemiDoc MP Imaging System with its built-in UV channel (Hercules, CA) for endpoint visual detection. The endpoint fluorescence was the determined raw fluorescence subtracting the averaged raw fluorescence of non-template control reaction. Specificity assay was investigated by using the control plasmids containing the complete N gene from SARS-CoV-2_PC, SARS-CoV control, MERS-CoV control, and Hs_RPP30_PC. Sensitivity assay was conducted by testing serially diluted SARS-CoV-2 RNA (Twist Bioscience) in water to concentrations of 5×10⁵, 5×10⁴, 5×10³, 5×10², and 5×10¹ copies/μl. CRISPR-Cas12a-based detection, namely trans-cleavage assay was incubated at 37° C. for 40 min and performed in a solution containing 1×isothermal amplification buffer, 0.32 μM A.s. Cas12a, 0.32 μM DAMP-crRNA, 1.0 μM synthetic SARS-CoV-2 N DNA fragment, and 1.0 μM ssDNA-FQ reporter.

### Digital WS-CRISPR Assay

The reaction system for digital WS-CRISPR assay was the same as that in tube-based bulk reaction mentioned above. The procedure of digital WS-CRISPR assay was modified based on the operational workflow for QuantStudio 3D digital PCR (Quick Reference Manual. Thermo Fisher Scientific). Briefly, a 15-μl digital WS-CRISPR reaction solution was first prepared in a tube by mixing Component A, B and the sample. Then, the reaction solution was loaded into the QuantStudio 3D digital PCR chip (version 2), followed by applying the lid, loading the immersion fluid, and sealing the chip. This step can be finished by using the QuantStudio 3D digital PCR Chip Loader (Thermo Fisher Scientific). Afterwards, the sealed chip was placed in ProFlex 2×Flat PCR System (Thermo Fisher Scientific) for 90-min incubation at 52° C. After incubation, the chip was taken out for the examination using a ZEISS Axio Observer fluorescence microscopy connected with ZEISS Axio Cam 305 and X-Cite 120Q fluorescence lamp illumination. For each chip's microscopy, the same parameters were set up including 5× magnification objective, 10× magnification eyepiece, 700 ms exposure time, 2.1 gamma value, and 2000 white value. Six distinct regions without overlapping areas were randomly captured by the microscopy to cover about 2809 microreactions. The number of positive spots was counted by using the ImageJ software. The step-by-step clicking after opening the images is Image>Type (8-bit)>Edit>Invert>Image>Adjust (Threshold: 0 and 245)>Apply>Analyze>Analyze Particles>Distribution>List. In the list, the count for over 0.02 bin start was enrolled and summed up.

Similarly, the specificity assay for the digital WS-CRISPR was finished by testing the control plasmids mentioned above and sensitivity was evaluated by testing serially diluted SARS-CoV-2 RNA (Twist Bioscience) in water to concentrations of 5×10⁵, 5×10⁴, 5×10³, 5×10², 5×10¹ and 5×10⁰ copies/μl, as well as 3×10⁶ copies/μl SARS-CoV-2 RNA extracted from a saliva sample. SARS-CoV-2 in each clinical sample was quantified using the calibration curve of digital WS-CRISPR by applying the averaged percentage of positive spots in six micrographs. Direct saliva digital WS-CRISPR assays were assessed by testing five saliva mock samples. The reaction system and procedure were the same as described above, replacing the target solution with 1.5 μl of heat-treated saliva mock sample solution at 95° C. for 5 min. The saliva mock samples (400 μl) were prepared by adding 1× inactivation reagent (0.0115 N NaOH, 1.0 mM EDTA, and 1.0 mM TCEP-HCl), 360 μl human saliva, and various volume percentages (0%, 1%, 2.5%, 5%, and 10%) of heat-inactivated SARS-CoV-2.

### Real-Time Quantitation RT-PCR Assay

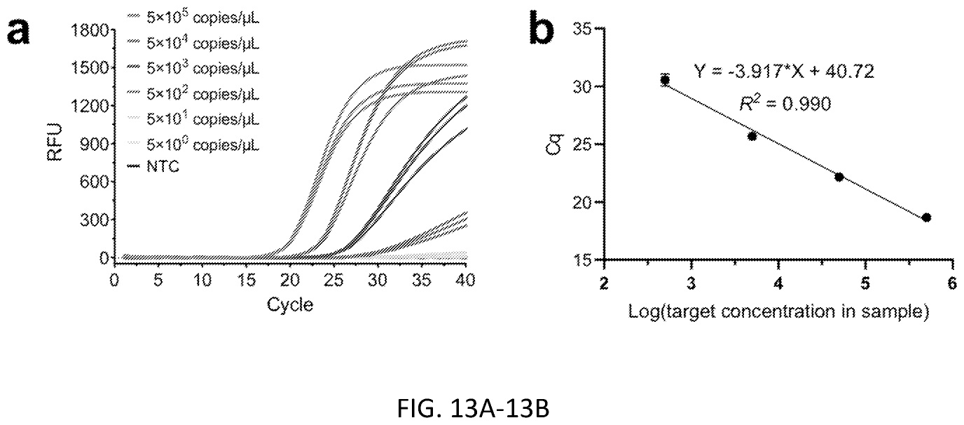

The real-time quantitation RT-PCR (RT-qCPR) assay for SARS-CoV-2 detection was carried out according to U.S. CDC 2019-Novel Coronavirus (2019-nCoV) Real-Time RT-PCR Diagnostic Panel (https://www.fda.gov/media/134922/download) with minor modifications. A typical RT-qPCR reaction system included 2 U/μl SuperScript IV reverse transcriptase, 1× QuantStudio master mix (Catalog #A26358), 0.5 μM nCOV_N1 forward primer, 0.5 μM nCOV_N1 reverse primer, 0.125 μM nCOV_N1 probe, and 1.5 μl of the target solution. The thermal cycler protocol consisted of Stage 1 (2.0 min at 25° C.), Stage 2 (15.0 min at 50° C.), Stage 3 (2.0 min at 95° C.) and Stage 4 (40 cycles of 3.0 s at 95° C. and 30 s at 55° C.). The capture point of fluorescence was set at 55° C. in Stage 4. Real-time quantitation analysis was performed in the Bio-Rad CFX96 Touch Real-Time PCR Detection System (Hercules, CA). Through testing serially diluted SARS-CoV-2 RNA (Twist Bioscience) with concentrations of 5×10⁵, 5×10⁴, 5×10³, and 5×10² copies/μl (FIG. 13A) a four-point calibration curve was plotted based on the relationship between Cq value and the log of target concentration (FIG. 13B). On the basis of this curve, SARS-CoV-2 in each clinical sample was quantified by loading the determined Cq value.

### Statistics and Reproducibility

GraphPad Software Prism 8.0.1 was used to plot real-time fluorescence curves, analyze linear regression, and verify statistical significances between two assay groups. The unpaired two-tailed t-test was made with the p value <0.05 as the threshold for defining significance. For endpoint imaging of the chip using fluorescence microscopy, six distinct regions without overlapping areas were randomly taken to cover about 2809 microreactions. Unless otherwise specified, each image for visual detection or micrograph for chip testing is a representative of at least three independent experiments. To plot the linear relationship between percentage of positive spots and concentration of targets in digital WS-CRISPR, total positive spots in all the six micrographs were used and three chips were taken to run three independent assays. Clinical sample testing by both digital WS-CRISPR and RT-qPCR assays was repeated three times to ensure data accuracy.

### REFERENCES

1. N. Zhu, D. Zhang, W. Wang, X. Li, B. Yang, J. Song, X. Zhao, B. Huang, W. Shi, R. Lu, A novel coronavirus from patients with pneumonia in China, 2019. New England Journal of Medicine, (2020).
2. WHO. https://covid19.who.int/. (2020).
3. W. Wang, Y. Xu, R. Gao, R. Lu, K. Han, G. Wu, W. Tan, Detection of SARS-CoV-2 in different types of clinical specimens. Jama 323, 1843-1844 (2020).
4. D. Wang, B. Hu, C. Hu, F. Zhu, X. Liu, J. Zhang, B. Wang, H. Xiang, Z. Cheng, Y. Xiong, Clinical characteristics of 138 hospitalized patients with 2019 novel coronavirus-infected pneumonia in Wuhan, China. Jama 323, 1061-1069 (2020).
5. C. Yan, J. Cui, L. Huang, B. Du, L. Chen, G. Xue, S. Li, W. Zhang, L. Zhao, Y. Sun, Rapid and visual detection of 2019 novel coronavirus (SARS-CoV-2) by a reverse transcription loop-mediated isothermal amplification assay. Clinical Microbiology and Infection, (2020).
6. L. Yu, S. Wu, X. Hao, X. Dong, L. Mao, V. Pelechano, W.-H. Chen, X. Yin, Rapid detection of COVID-19 coronavirus using a reverse transcriptional loop-mediated isothermal amplification (RT-LAMP) diagnostic platform. Clin. Chem., (2020).
7. B. A. Rabe, C. Cepko, SARS-CoV-2 detection using isothermal amplification and a rapid, inexpensive protocol for sample inactivation and purification. Proceedings of the National Academy of Sciences 117, 24450-24458 (2020).
8. O. Behrmann, I. Bachmann, M. Spiegel, M. Schramm, A. A. El Wahed, G. Dobler, G. Dame, F. T. Hufert, Rapid detection of SARS-CoV-2 by low volume real-time single tube reverse transcription recombinase polymerase amplification using an exo probe with an internally linked quencher (exo-IQ). Clin. Chem., (2020).
9. S. Xia, X. Chen, Single-copy sensitive, field-deployable, and simultaneous dual-gene detection of SARS-CoV-2 RNA via modified RT-RPA. Cell Discovery 6, 1-4 (2020).
10. G. Xue, S. Li, W. Zhang, B. Du, J. Cui, C. Yan, L. Huang, L. Chen, L. Zhao, Y. Sun, Reverse-Transcription Recombinase-Aided Amplification Assay for Rapid Detection of the 2019 Novel Coronavirus (SARS-CoV-2). Anal. Chem. 92, 9699-9705 (2020).
11. J. Wang, K. Cai, X. He, X. Shen, J. Wang, J. Liu, J. Xu, F. Qiu, W. Lei, L. Cui, A multiple center clinical evaluation of an ultra-fast single-tube assay for SARS-CoV-2 RNA. Clinical Microbiology and Infection, (2020).
12. C. H. Woo, S. Jang, G. Shin, G. Y. Jung, J. W. Lee, Sensitive fluorescence detection of SARS-CoV-2 RNA in clinical samples via one-pot isothermal ligation and transcription. Nature biomedical engineering, 1-12 (2020).
13. D. S. Chertow, Next-generation diagnostics with CRISPR. Science 360, 381-382 (2018).
14. S.-Y. Li, Q.-X. Cheng, J.-K. Liu, X.-Q. Nie, G.-P. Zhao, J. Wang, CRISPR-Cas12a has both cis- and trans-cleavage activities on single-stranded DNA. Cell research 28, 491-493 (2018).
15. O. O. Abudayyeh, J. S. Gootenberg, S. Konermann, J. Joung, I. M. Slaymaker, D. B. Cox, S. Shmakov, K. S. Makarova, E. Semenova, L. Minakhin, C2c2 is a single-component programmable RNA-guided RNA-targeting CRISPR effector. Science 353, aaf5573 (2016).
16. J. S. Chen, E. Ma, L. B. Harrington, M. Da Costa, X. Tian, J. M. Palefsky, J. A. Doudna, CRISPR-Cas12a target binding unleashes indiscriminate single-stranded DNase activity. Science 360, 436-439 (2018).
17. J. P. Broughton, X. Deng, G. Yu, C. L. Fasching, V. Servellita, J. Singh, X. Miao, J. A. Streithorst, A. Granados, A. Sotomayor-Gonzalez, CRISPR-Cas12-based detection of SARS-CoV-2. Nat. Biotechnol., 1-5 (2020).
18. M. Patchsung, K. Jantarug, A. Pattama, K. Aphicho, S. Suraritdechachai, P. Meesawat, K. Sappakhaw, N. Leelahakorn, T. Ruenkam, T. Wongsatit, Clinical validation of a Cas13-based assay for the detection of SARS-CoV-2 RNA. Nature Biomedical Engineering, 1-10 (2020).
19. X. Ding, K. Yin, Z. Li, R. V. Lalla, E. Ballesteros, M. M. Sfeir, C. Liu, Ultrasensitive and visual detection of SARS-CoV-2 using all-in-one dual CRISPR-Cas12a assay. Nature communications 11, 1-10 (2020).
20. J. Joung, A. Ladha, M. Saito, N.-G. Kim, A. E. Woolley, M. Segel, R. P. Barretto, A. Ranu, R. K. Macrae, G. Faure, Detection of SARS-CoV-2 with SHERLOCK One-Pot Testing. New England Journal of Medicine 383, 1492-1494 (2020).
21. X. Wu, C. Chan, Y. H. Lee, S. L. Springs, T. K. Lu, H. Yu, A Digital CRISPR-based Method for the Rapid Detection and Absolute Quantification of Viral Nucleic Acids. medRxiv, (2020).
22. E.-C. Yeh, C.-C. Fu, L. Hu, R. Thakur, J. Feng, L. P. Lee, Self-powered integrated microfluidic point-of-care low-cost enabling (SIMPLE) chip. Science advances 3, e1501645 (2017).
23. X. Ding, Z. Xu, K. Yin, M. Sfeir, C. Liu, Dual-Priming isothermal amplification (DAMP) for highly sensitive and specific molecular detection with ultralow nonspecific signals. Anal. Chem. 91, 12852-12858 (2019).
24. T. Guanghui, J. Gong, L. Kan, X. Zhang, Y. He, J. Pan, L. Zhao, J. Tian, S. Lin, Z. Lu, A general onepot-method for nucleic acid detection with CRISPR-Cas12a. (2020).
25. L. T. Nguyen, B. M. Smith, P. K. Jain, Enhancement of trans-cleavage activity of Cas12a with engineered crRNA enables amplified nucleic acid detection. bioRxiv, (2020).
26. R. T. Fuchs, J. Curcuru, M. Mabuchi, P. Yourik, G. B. Robb, Cas12a trans-cleavage can be modulated in vitro and is active on ssDNA, dsDNA, and RNA. bioRxiv, 600890 (2019).
27. M. Jinek, K. Chylinski, I. Fonfara, M. Hauer, J. A. Doudna, E. Charpentier, A programmable dual-RNA-guided DNA endonuclease in adaptive bacterial immunity. science 337, 816-821 (2012).
28. T. Notomi, H. Okayama, H. Masubuchi, T. Yonekawa, K. Watanabe, N. Amino, T. Hase, Loop-mediated isothermal amplification of DNA. Nucleic Acids Res. 28, e63-e63 (2000).
29. S. Cai, C. Jung, S. Bhadra, A. D. Ellington, Phosphorothioated primers lead to loop-mediated isothermal amplification at low temperatures. Anal. Chem. 90, 8290-8294 (2018).
30. R. Sanders, J. F. Huggett, C. A. Bushell, S. Cowen, D. J. Scott, C. A. Foy, Evaluation of digital PCR for absolute DNA quantification. Anal. Chem. 83, 6474-6484 (2011).
31. A. S. Whale, J. F. Huggett, S. Cowen, V. Speirs, J. Shaw, S. Ellison, C. A. Foy, D. J. Scott, Comparison of microfluidic digital PCR and conventional quantitative PCR for measuring copy number variation. Nucleic Acids Res. 40, e82-e82 (2012).
32. A. L. Wyllie, J. Fournier, A. Casanovas-Massana, M. Campbell, M. Tokuyama, P. Vijayakumar, J. L. Warren, B. Geng, M. C. Muenker, A. J. Moore, Saliva or nasopharyngeal swab specimens for detection of SARS-CoV-2. New England Journal of Medicine 383, 1283-1286 (2020).
33. J. Strecker, S. Jones, B. Koopal, J. Schmid-Burgk, B. Zetsche, L. Gao, K. S. Makarova, E. V. Koonin, F. Zhang, Engineering of CRISPR-Cas12b for human genome editing. Nature communications 10, 1-8 (2019).
34. F. Teng, L. Guo, T. Cui, X.-G. Wang, K. Xu, Q. Gao, Q. Zhou, W. Li, CDetection: CRISPR-Cas12b-based DNA detection with sub-attomolar sensitivity and single-base specificity. Genome biology 20, 1-7 (2019).
35. Q. Wei, R. Nagi, K. Sadeghi, S. Feng, E. Yan, S. J. Ki, R. Caire, D. Tseng, A. Ozcan, Detection and spatial mapping of mercury contamination in water samples using a smart-phone. ACS nano 8, 1121-1129 (2014).
36. Q. Wei, H. Qi, W. Luo, D. Tseng, S. J. Ki, Z. Wan, Z. n. Göröcs, L. A. Bentolila, T.-T. Wu, R. Sun, Fluorescent imaging of single nanoparticles and viruses on a smart phone. ACS nano 7, 9147-9155 (2013).
37. T. Gou, J. Hu, W. Wu, X. Ding, S. Zhou, W. Fang, Y. Mu, Smartphone-based mobile digital PCR device for DNA quantitative analysis with high accuracy. Biosens. Bioelectron. 120, 144-152 (2018).
38. X. Ding, K. Nie, L. Shi, Y. Zhang, L. Guan, D. Zhang, S. Qi, X. Ma, Improved detection limit in rapid detection of human enterovirus 71 and coxsackievirus A16 by a novel reverse transcription-isothermal multiple-self-matching-initiated amplification assay. J. Clin. Microbiol. 52, 1862-1870 (2014).
39. L. Cui, Y. Ge, X. Qi, G. Xu, H. Li, K. Zhao, B. Wu, Z. Shi, X. Guo, L. Hu, Detection of severe fever with thrombocytopenia syndrome virus by reverse transcription-cross-priming amplification coupled with vertical flow visualization. J. Clin. Microbiol. 50, 3881-3885 (2012).

1. A digital warm-start assay for detecting a target in a sample, assay comprising:
a. contacting a sample with a warm-start CRISPR reaction mixture, the warm-start CRISPR reaction mixture comprising a Cas endonuclease, a target-specific crRNA, outer primers for amplification of the target, phosphorothioated inner primers for amplification of the target, and pyrophosphatase;
b. partitioning the warm-start CRISPR reaction mixture into a plurality of microwells;
c. amplifying the target, if present in the sample, by isothermal amplification; and
d. detecting a signal in each of the plurality of microwells, wherein detection of the signal in a given microwell indicates the presence of the target in the microwell.

2. A digital warm-start assay for detecting a target in a sample, assay comprising:
a. providing a warm-start CRISPR reaction mixture, the warm-start CRISPR reaction mixture comprising a Cas endonuclease, a target-specific crRNA, outer primers for isothermal amplification of the target, phosphorothioated inner primers for isothermal amplification of the target, and pyrophosphatase;
b. partitioning the warm-start CRISPR reaction mixture into a plurality of microwells, wherein each of the plurality of microwells comprises a sample;
c. amplifying the target, if present in the sample, by isothermal amplification; and
d. detecting a signal in each of the plurality of microwells, wherein a detectable signal in a given microwell indicates the presence of the target in the microwell.

3. The method of claim 1 or claim 2, wherein the Cas endonuclease comprises Cas12a.

4. The method of any one of the preceding claims, wherein the warm-start CRISPR reaction mixture further comprises a forward competition primer and a reverse competition primer.

5. The method of any one of the preceding claims, wherein the warm-start CRISPR reaction mixture further comprises a DNA polymerase.

6. The method of claim 5, wherein the DNA polymerase comprises Bst DNA polymerase.

7. The method of any one of the preceding claims, wherein the warm-start CRISPR reaction mixture further comprises a reverse transcriptase.

8. The method any one of the preceding claims, wherein isothermal amplification comprises incubating the warm-start CRISPR reaction mixture at a temperature of about 50° C.-60° C. for at least about 10 minutes.

9. The method of claim 8, comprising incubating for at least about 30 minutes.

10. The method of claim 8, comprising incubating for at least about 60 minutes.

11. The method of claim 8, comprising incubating for about 90 minutes.

12. The method of any one of claims 8-11, wherein isothermal amplification comprises incubating the warm-start CRISPR reaction mixture at a temperature of about 52° C.

13. The method of any one of the preceding claims, wherein the target is viral nucleic acid.

14. The method of claim 13, wherein the target is viral nucleic acid from an upper respiratory pathogen selected from SARS-CoV-2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, Aspergillus, and Streptococcus pyogenes.

15. The method of claim 14, wherein the target is SARS-CoV-2 nucleic acid.

16. The method of claim 15, wherein the target is nucleic acid within the nucleoprotein (N) gene of SARS-CoV-2.

17. The method of claim 16, wherein the target-specific crRNA comprises a nucleotide sequence having at least 90% sequence identity to SEQ ID NO: 9.

18. The method of claim 17, wherein the target-specific crRNA comprises a nucleotide sequence set forth in SEQ ID NO: 9.

19. The method of any one of the preceding claims, wherein the outer primers comprise a forward outer primer and a reverse outer primer, wherein the forward outer primer comprises a nucleotide sequence having at least 90% sequence identity to SEQ ID NO: 1 and wherein the reverse outer primer comprises a nucleotide sequence having at least 90% sequence identity to SEQ ID NO: 2.

20. The method of claim 19, wherein the forward outer primer comprises a nucleotide sequence set forth in SEQ ID NO: 1 and wherein the reverse outer primer comprises a nucleotide sequence set forth in SEQ ID NO: 2.

21. The method of any one of the preceding claims, wherein the phosphorothioated inner primers comprise a forward inner primer and a reverse inner primer, wherein the forward inner primer comprises a nucleotide sequence having at least 90% sequence identity to SEQ ID NO: 4, and wherein the reverse inner primer comprises a nucleotide sequence having at least 90% sequence identity to SEQ ID NO: 6.

22. The method of claim 21, wherein the forward inner primer comprises a nucleotide sequence set forth in SEQ ID NO: 4, and wherein the reverse inner primer comprises a nucleotide sequence set forth in SEQ ID NO: 6.

23. The method of any one of the preceding claims, wherein the warm-start CRISPR reaction mixture comprises less than 0.4 U/μl pyrophosphatase.

24. The method of claim 23, wherein the warm-start CRISPR reaction mixture comprises about 0.2 U/μl pyrophosphatase.

25. The method of any one of the preceding claims, wherein the sample comprises a nasal sample, a nasopharyngeal sample, an oropharyngeal sample, a mid-turbinate sample, or a saliva sample.

26. The method of any one of the preceding claims, wherein the sample is obtained from a subject suspected having a viral infection.

27. The method of claim 26, wherein the sample is obtained from a subject suspected of having an upper respiratory infection.

28. The method of claim 27, wherein the sample is obtained from a subject suspected of having infection with an upper respiratory pathogen selected from SARS-CoV-2, coronavirus, rhinovirus, influenza, respiratory syncytial virus, adenovirus, parainfluenza, human immunodeficiency virus, human papillomavirus, rotavirus, hepatitis C virus, zika virus, Ebola virus, tuberculosis, Borrelia burgdorferi, Staphylococcus, Aspergillus, and Streptococcus pyogenes.

29. The method of claim 28, wherein the sample is obtained from a subject suspected of having SARS-CoV-2.

30. A kit comprising a primer having at least 90% sequence identity to SEQ ID NO: 1, a primer having at least 90% sequence identity to SEQ ID NO: 2, a primer having at least 90% sequence identity to SEQ ID NO: 4, a primer having at least 90% sequence identity to SEQ ID NO: 6, a primer having at least 90% sequence identity to SEQ ID NO: 7, and a primer having at least 90% sequence identity to SEQ ID NO: 8.

31. The kit of claim 30, comprising a primer having the amino acid sequence set forth in SEQ ID NO: 1, a primer having the amino acid sequence set forth in SEQ ID NO: 2, a primer having the amino acid sequence set forth in SEQ ID NO: 4, a primer having the amino acid sequence set forth in SEQ ID NO: 6, a primer having the amino acid sequence set forth in SEQ ID NO: 7, and a primer having the amino acid sequence set forth in SEQ ID NO: 8.

32. The kit of claim 30 or claim 31, further comprising a target-specific crRNA comprising a nucleotide sequence having at least 90% sequence identity to SEQ ID NO: 9.

33. The kit of claim 32, further comprising a target specific crRNA comprising the nucleotide sequence set forth in SEQ ID NO: 9.

34. The kit of any one of claims 30-34, further comprising pyrophosphatase.


SEQUENCE LISTING (18 sequences)

SEQ ID NO: 1 - DNA, 19 bp, Artificial sequence
  Synthetic
ggcttctacgcagaaggga

SEQ ID NO: 2 - DNA, 19 bp, Artificial sequence
  Synthetic
aatctgtcaagcagcagca

SEQ ID NO: 3 - DNA, 45 bp, Artificial sequence
  Synthetic
ttgaactgttgcgactacgtgttttagcctcttctcgttcctcat

SEQ ID NO: 4 - DNA, 45 bp, Artificial sequence; identical to SEQ ID NO: 3
  Synthetic

SEQ ID NO: 5 - DNA, 44 bp, Artificial sequence
  Synthetic
gaacttctcctgctagaatggcttttgcaagagcagcatcaccg

SEQ ID NO: 6 - DNA, 44 bp, Artificial sequence; identical to SEQ ID NO: 5
  Synthetic

SEQ ID NO: 7 - DNA, 21 bp, Artificial sequence
  Synthetic
ttgaactgttgcgactacgtg

SEQ ID NO: 8 - DNA, 22 bp, Artificial sequence
  Synthetic
gaacttctcctgctagaatggc

SEQ ID NO: 9 - RNA, 44 nt, Artificial sequence
  Synthetic
uaauuucuacuaaguguagaucccuacugcugccuggaguugaa

SEQ ID NO: 10 - DNA, 70 bp, Artificial sequence
  Synthetic
gccattcgagcaggagaatttcccctactgctgccaggagttgaatttcttgaattaccgcgactacgtg

SEQ ID NO: 11 - DNA, 20 bp, Artificial sequence
  Synthetic
gaccccaaaatcagcgaaat

SEQ ID NO: 12 - DNA, 24 bp, Artificial sequence
  Synthetic
tctggttactgccagttgaatctg

SEQ ID NO: 13 - DNA, 24 bp, Artificial sequence
  Synthetic
accccgcattacgtttggtggacc

SEQ ID NO: 14 - DNA, 33 bp, Artificial sequence
  Synthetic
aggcagcagtaggggaacttctcctgctagaat

SEQ ID NO: 15 - DNA, 33 bp, Artificial sequence
  Synthetic
ttggcctttaccagacattttgctctcaagctg

SEQ ID NO: 16 - RNA, 41 nt, Artificial sequence
  Synthetic
uaauuucuacuaaguguagaucaucaccgccauugccagcc

SEQ ID NO: 17 - RNA, 41 nt, Artificial sequence
  Synthetic
uaauuucuacuaaguguagauuugcugcugcuugacagauu

SEQ ID NO: 18 - DNA, 5 bp, Artificial sequence
  Synthetic
ccccc